(19) United States
(12) Patent Application Publication
(10) Pub. No.: US 2026/0258072 A1
(43) Pub. Date: Sep. 3, 2026

(54) PROCESS AND INTERMEDIATES FOR THE PREPARATION OF 3ALPHA-HYDROXY-3BETA-ALKYL STEROIDS

(71) Applicant: CURIA SPAIN, S.A.U., Boecillo, Valladolid (ES)

(72) Inventors: María MARCO ARIAS, Boecillo, Valladolid (ES); Ana GIL ORDÓÑEZ, Boecillo, Valladolid (ES); Ivano MESSINA, Boecillo, Valladolid (ES)

(21) Appl. No.: 19/164,767

(22) PCT Filed: Mar. 13, 2024
(86) PCT No.: PCT/EP2024/056622

§ 371 Date: Sep. 12, 2025

(30) Foreign Application Priority Data
Mar. 14, 2023 (EP) 23382238.6

(51) Int. Cl. C07J 1/00; C07J 13/00; C07J 43/00

(52) U.S. Cl. CPC C07J 1/0059; C07J 13/007; C07J 43/003

(57) ABSTRACT

The invention relates to a process for the preparation of 3α-hydroxy-3β-alkyl steroids, such as Zuranolone and structurally related compounds, which comprises the use of a compound of formula (I), or a salt or solvate thereof, as intermediate:

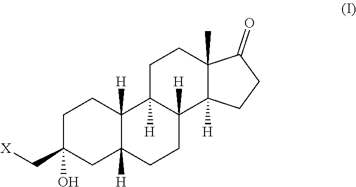

wherein X is selected from Cl, Br and I.

The invention also relates to a process for preparing a compound of formula (I), or a salt or solvate thereof.

### FIELD OF THE INVENTION

The invention relates to new intermediates useful in the preparation of 3α-hydroxy-3β-alkyl steroids, such as zuranolone and structurally related compounds, and to a process for preparing said intermediates.

### BACKGROUND OF THE INVENTION

Zuranolone and other related 3α-hydroxy-3β-alkyl steroids have been disclosed in the prior art as neuroactive compounds and, therefore, useful in the prevention and treatment of CNS-related diseases.

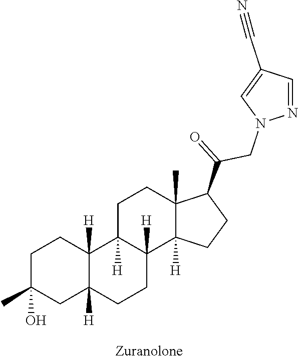

Several synthetic processes for the preparation of this type of compounds and intermediates thereof have been disclosed. In particular, different methods for the introduction of an alkyl group and a hydroxyl group at position 3 of the steroid have been described; however, most of them do not provide the desired product (α-hydroxy-β-alkyl product) in a stereoselective manner.

WO2013/056181 discloses a process comprising protection of the ketone at position 3 before olefination of the ketone at position 17. Subsequent deprotection of the 3-ketone and MeMgBr addition result in the 3-hydroxy-, 3-methyl-functionality though with very low selectivity for the desired isomer (an alpha/beta ratio of the hydroxyl group of about 25/75 (6b/6a) after chromatographic purification is disclosed in example 1).

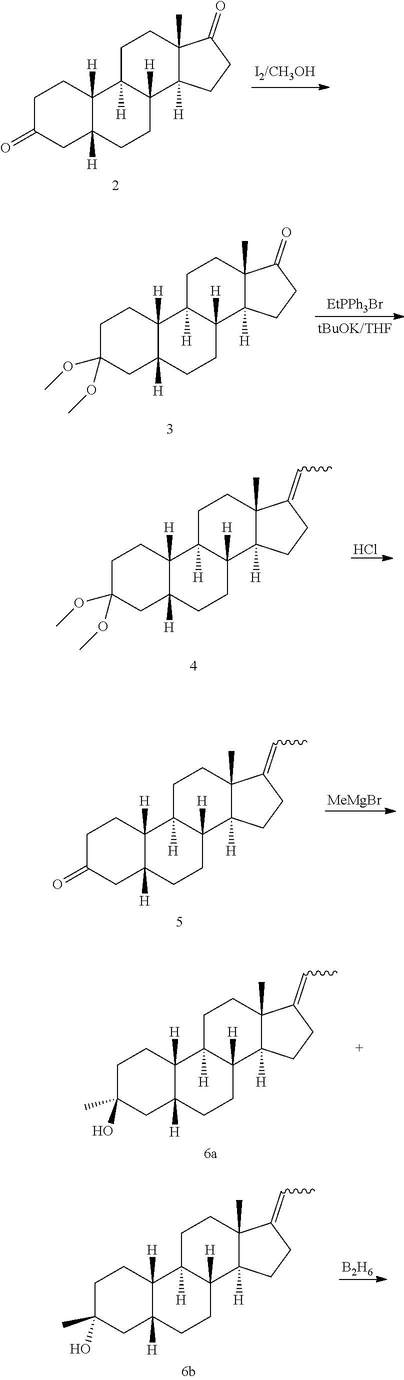

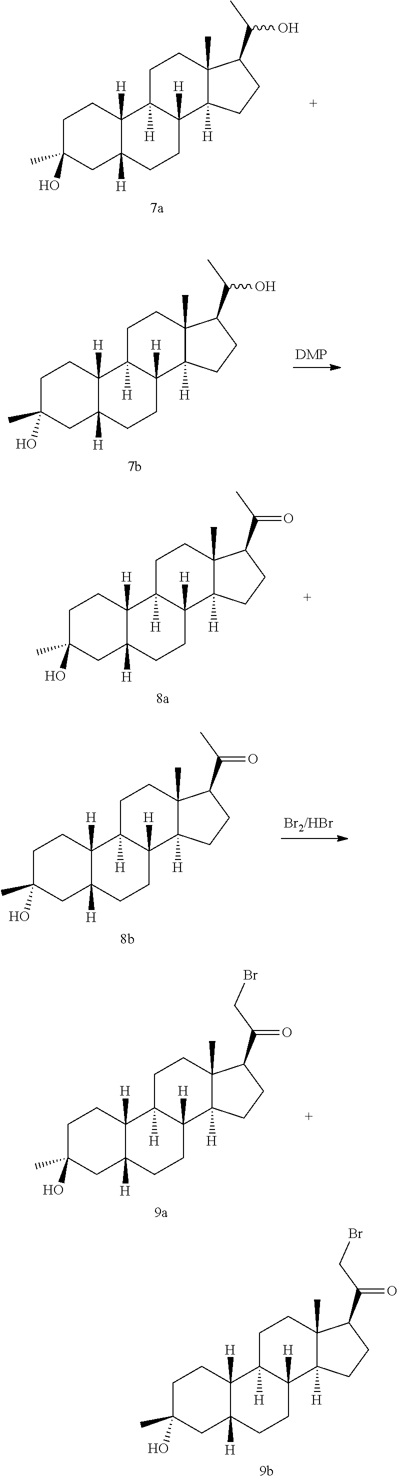

This document discloses other strategies for the functionalization at position 3, such as addition of a fluorinated sulfone, but again low selectivity is obtained (an alpha/beta ratio of the hydroxyl group of about 57/43 (11b/11a) after chromatographic purification is disclosed in example 2).

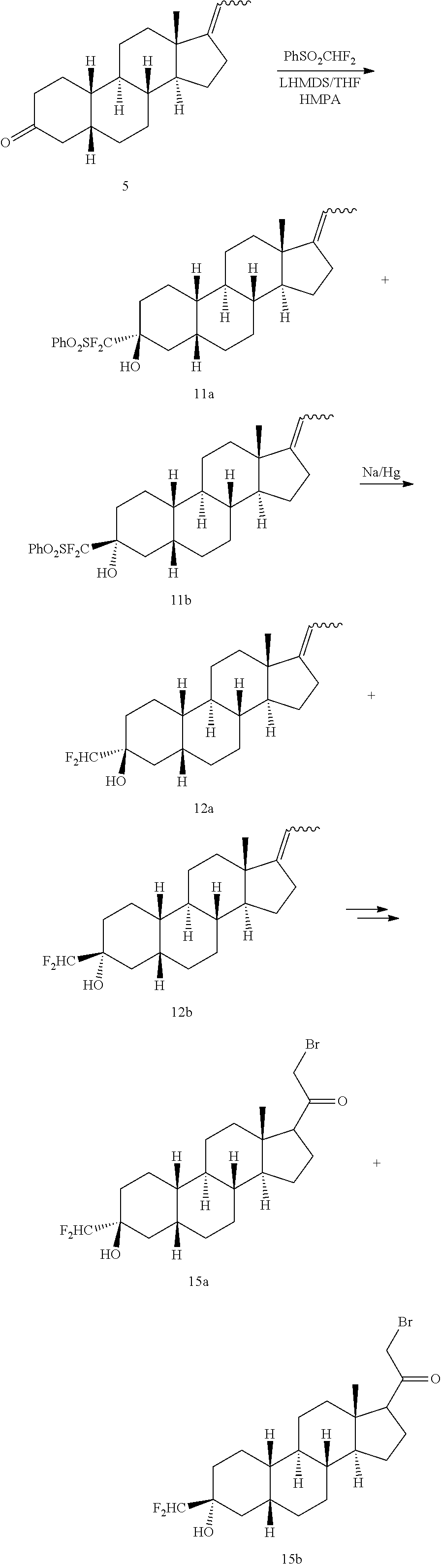

Compounds of formula 9a/9b and 15a/15b in WO2013/056181 are then converted into neuroactive steroids by reaction with different heterocyclic and heteroaryl compounds.

WO2014/169832 discloses a process for the synthesis of key intermediate SA where the 3-hydroxy,3-methyl functionality is obtained by addition of MeMgBr to the 3-ketone in the presence of MAD (methylaluminum bis(2,6-di-tert-butyl-4-methylphenoxide)).

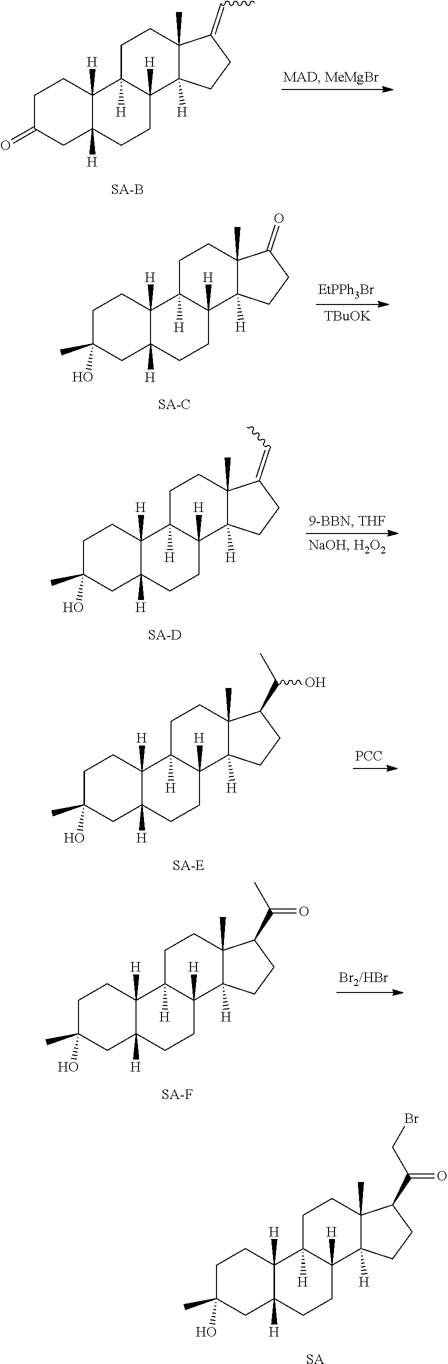

In this case, the stereoselectivity in the MeMgBr addition is achieved by the use of MAD. However, three equivalents of MAD are needed and, furthermore, MAD is an expensive and difficult to handle reagent that has to be prepared in situ by reaction of trimethyl aluminum (pyrophoric) with 2,6-di-tert-butyl-4-methylphenol. Accordingly, this method would not be suitable for industrial production.

Additionally, the inventors of the present invention did not obtain good results when they tried to reproduce this strategy (comparative example 3).

A similar strategy, using MeMgBr and MAD is disclosed in WO2016/061527.

WO2014/169832 and WO2014/169836 disclose the preparation of 3-hydroxy,3-fluoromethyl derivatives through epoxide opening. However, the epoxide is formed with poor stereoselectivity and the corresponding isomers are separated at the end of the synthesis in a ratio of about 50/50.

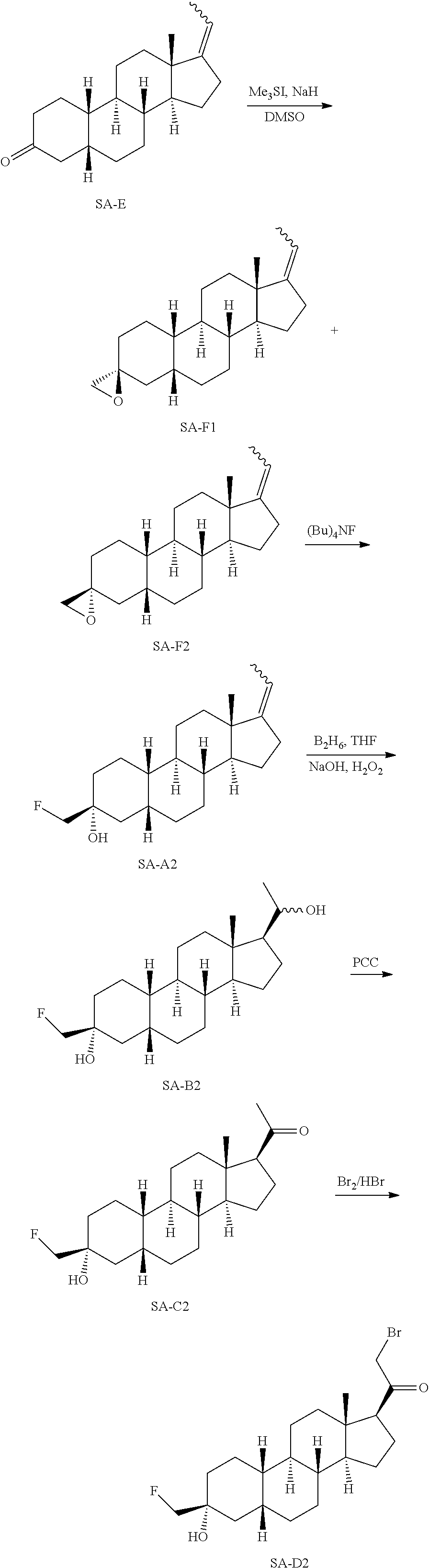

WO2014/169832 also describes compounds with a vinyl group at position 17 (compounds of formula SA-G) as useful intermediates in the preparation of active steroids.

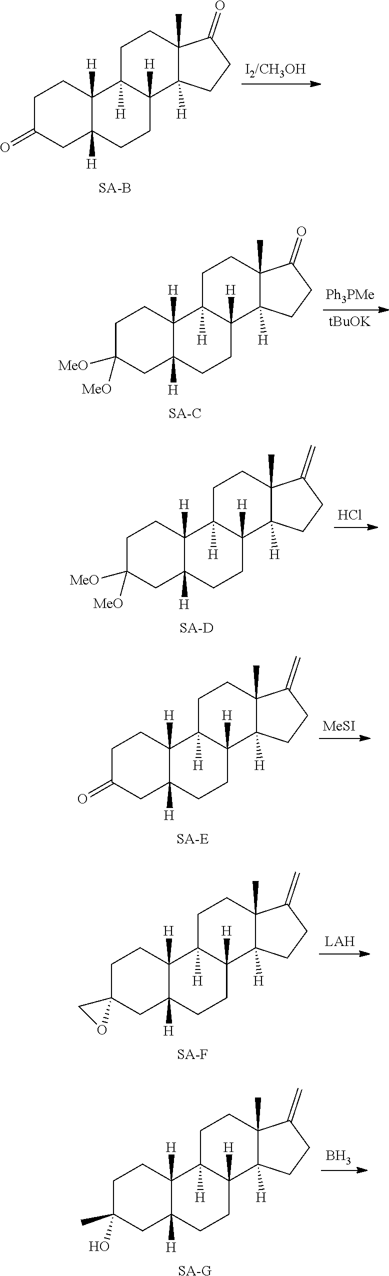

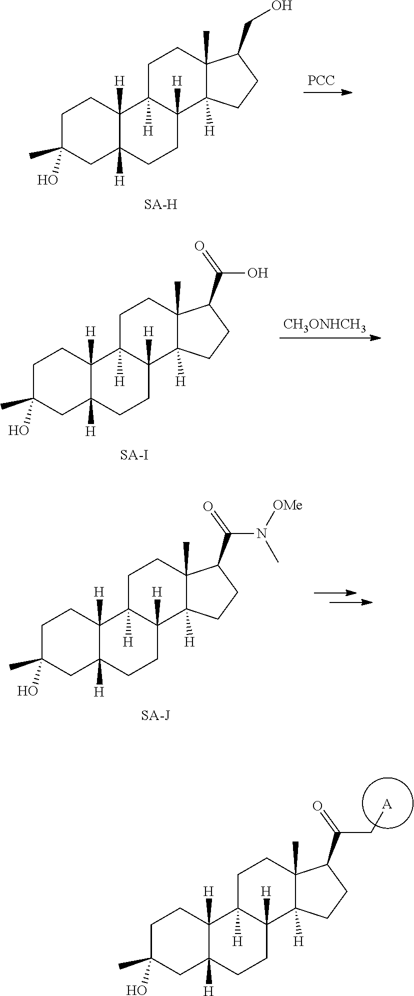

WO2014/169833 and WO2015/180679 disclose the preparation of 3-hydroxy,3-alkoxymethyl derivatives through epoxide opening with MeONa or EtONa, respectively. However, the epoxide is formed with poor stereoselectivity and the corresponding isomers are separated at the end of the synthesis in a ratio of about 50/50.

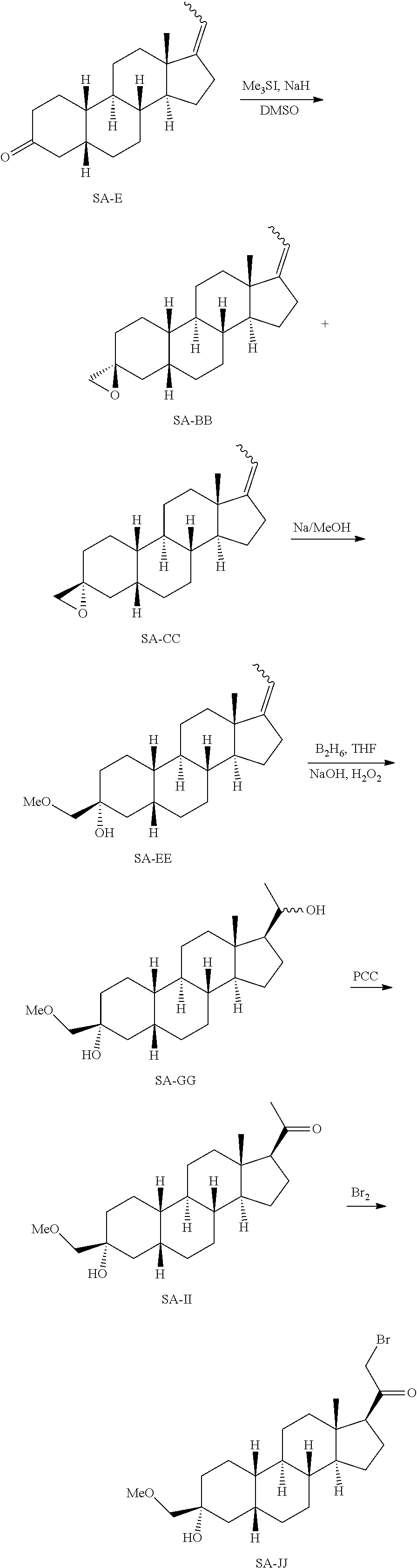

WO2014/169833 describes the preparation of 3α-hydroxy,3ρ-ethoxymethyl derivatives in a stereoselective manner for steroids where the two connected cyclohexane rings are in the form of a trans-decaline, as in compounds SB below.

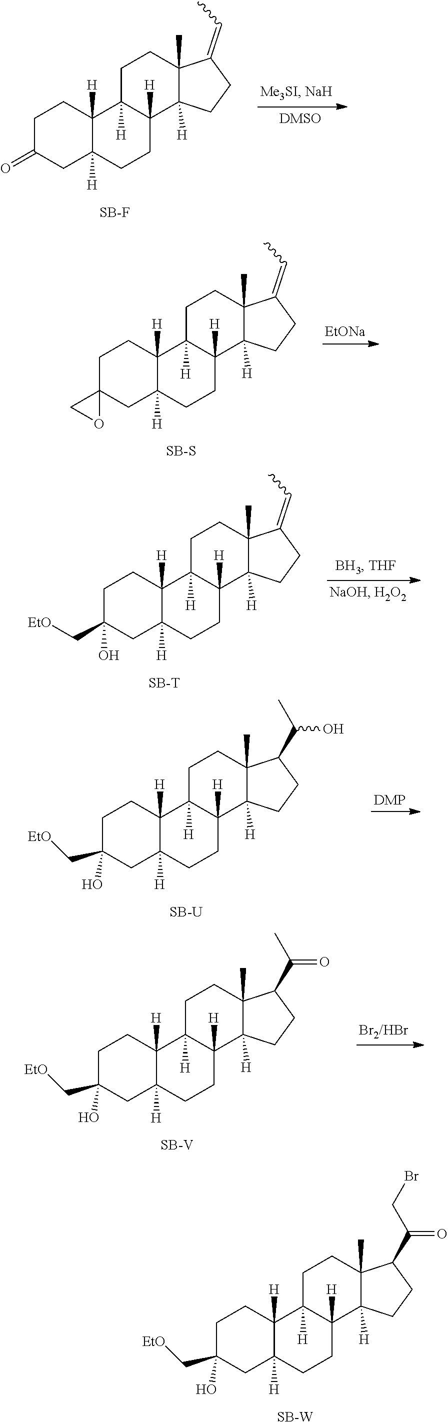

However, as shown in WO2020/118060, this strategy results in low stereoselectivity when applied to cis-decalins (as compounds 87 below). In particular, this document discloses the synthesis of 3-hydroxy,3-alkoxymethyl derivatives through epoxidation of the 3,17-diketone and subsequent epoxide opening with the sodium alkoxide; however, a ratio of isomers at position 3 of about 71/29 is disclosed in example 87 for compound 87.3.

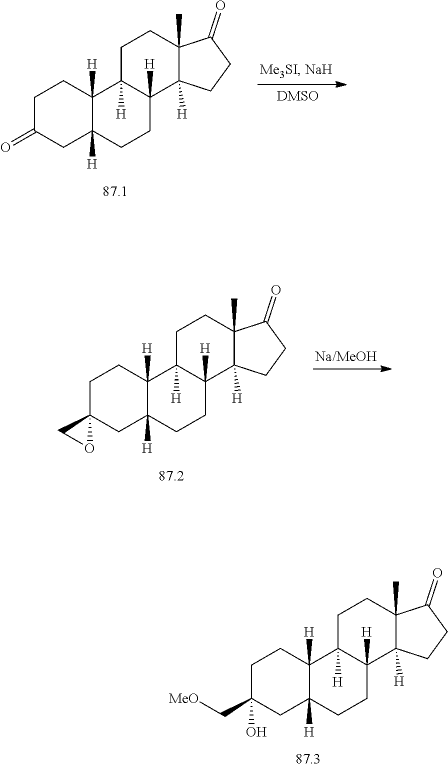

WO2020/118060 also describes compounds with a vinyl group at position 17 (compounds of formula A34) as useful intermediates in the preparation of active steroids.

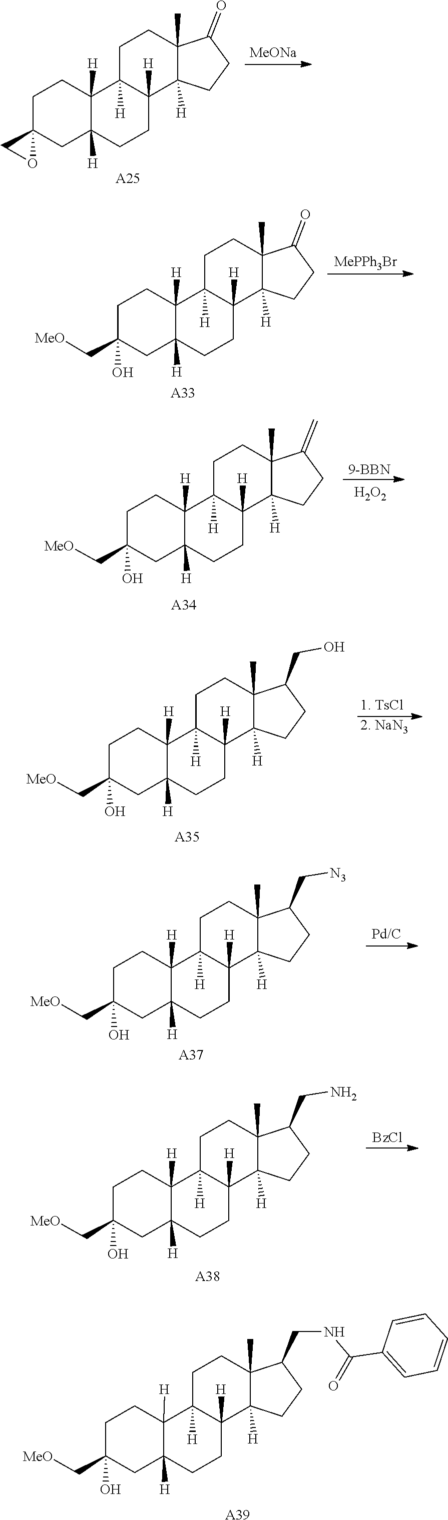

Despite the methods in the prior art, it is still necessary to develop new processes for preparing 3α-hydroxy-3β-alkyl steroids, such as Zuranolone, as well as key intermediates in their synthesis, which overcome all or part of the problems associated with the known processes belonging to the state of the art.

### SUMMARY OF THE INVENTION

The invention faces the problem of providing new methods for the preparation of α-hydroxy-β-alkyl steroids and intermediates thereof.

In particular, the inventors have found that compounds of formula (I) can be obtained by halohydrin formation reaction of a compound of formula (III). This reaction proceeds in a stereoselective manner. Further, compounds of formula (III) can in turn be obtained by olefination of a compound of formula (II). This olefination proceeds in a regioselective manner, so that protection/deprotection steps are not required. Due to the regioselectivity of the olefination reaction and the stereoselectivity of the halohydrin formation reaction, this synthetic strategy results in a very straightforward and effective process for the preparation of compounds of formula (I) and, therefore, of 3α-hydroxy-3β-alkyl steroids.

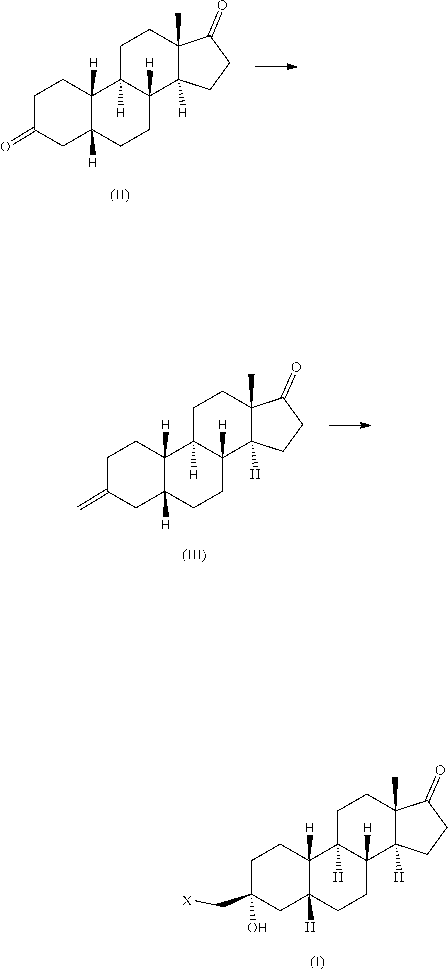

Additionally, the compound of formula (I) is a very versatile intermediate since the group X can be easily converted into other functional groups. Therefore, the compound of formula (I) can be used as a common key intermediate in the manufacture of different steroids.

Accordingly, in a first aspect the invention is directed to a process for preparing a compound of formula (I) or a salt or solvate thereof

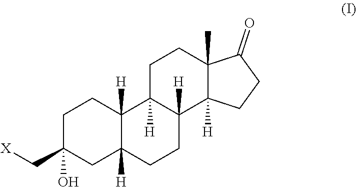

wherein X is selected from Cl, Br and I;
   which comprises
   (a) olefination of a compound of formula (II) or a salt or solvate thereof

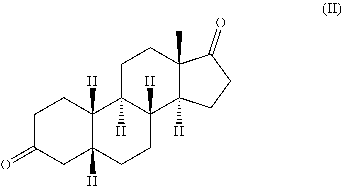

to provide a compound of formula (III) or a salt or solvate thereof

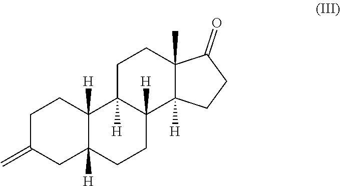

and
   (b) halohydrin formation reaction of the compound of formula (III), or a salt or solvate thereof, to provide the compound of formula (I), or a salt or solvate thereof.

In a second aspect the invention is directed to a process for preparing a compound of formula (IX) or a salt or solvate thereof

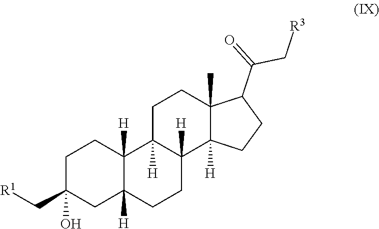

wherein
R¹ is selected from H, F, C₁₋₆ alkyl, C₁₋₆ alkoxyl, C₁₋₆ haloalkyl, and N(R′)₂, wherein each R′ is independently selected from H and C₁₋₆ alkyl; and
R³ is selected from 5- to 10-membered heterocyclyl and 5- to 10-membered heteroaryl, wherein the 5- to 10-membered heterocyclyl and the 5- to 10-membered heteroaryl are unsubstituted or substituted with a substituent selected from the group consisting of C₁₋₆ alkyl, C₁₋₆ haloalkyl, halogen, —CN, NO₂, —N(Rₐ)(R_{b}), —OR_{c}, —SR_{d}, —C(O)Rₑ, —C(O)OR_{f}, —C(O)N(R_{g})(Rₕ) and —OC(O)Rᵢ; wherein Rₐ, R_{b}, R_{c}, R_{d}, Rₑ, R_{f}, R_{g}, Rₕ and Rᵢ are independently selected from hydrogen, C₁-C₆ alkyl and C₁-C₆ haloalkyl;
which comprises
(a) olefination of a compound of formula (II) or a salt or solvate thereof

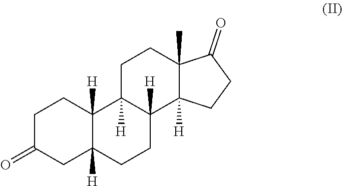

to provide a compound of formula (III) or a salt or solvate thereof

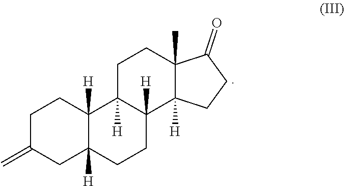

(b) halohydrin formation reaction of the compound of formula (III), or a salt or solvate thereof, to provide a compound of formula (I), or a salt or solvate thereof.

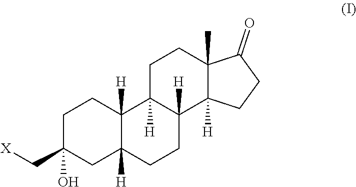

wherein X is selected from Cl, Br and I;
(c) conversion of the compound of formula (I), or a salt or solvate thereof, into a compound of formula (IV), or a salt or solvate thereof

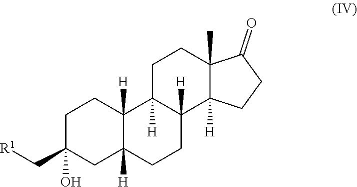

(d) olefination of the compound of formula (IV), or a salt or solvate thereof, to provide a compound of formula (Va), or a salt or solvate thereof

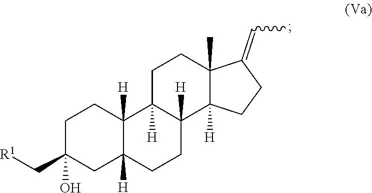

(e) hydroxylation of the compound of formula (Va), or a salt or solvate thereof, to provide a compound of formula (VI), or a salt or solvate thereof

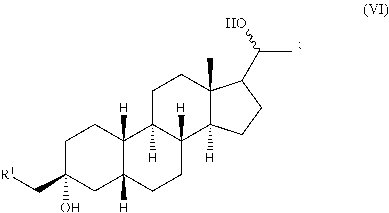

(f) oxidation of the compound of formula (VI), or a salt or solvate thereof, to provide a compound of formula (VII), or a salt or solvate thereof

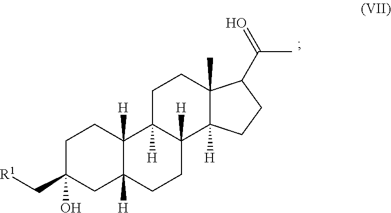

(g) halogenation of the compound of formula (VII), or a salt or solvate thereof, to provide a compound of formula (VIII), or a salt or solvate thereof

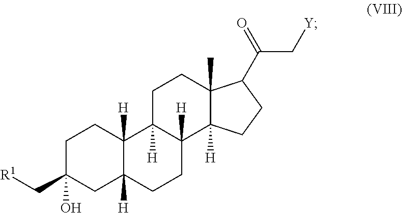

wherein Y is halogen; and
(h) reaction of the compound of formula (VIII), or a salt or solvate thereof, with a 5- to 10-membered heterocycle or a 5- to 10-membered heteroaryl, wherein the 5- to 10-membered heterocycle and the 5- to 10-membered heteroaryl are unsubstituted or substituted with a substituent selected from the group consisting of C₁₋₆ alkyl, C₁₋₆ haloalkyl, halogen, —CN, NO₂, —N(Rₐ)(R_{b}), —OR_{c}, —SR_{d}, —C(O)Rₑ, —C(O)OR_{f}, —C(O)N(R_{g})(Rₕ) and —OC(O)Rᵢ; wherein Rₐ, R_{b}, R_{c}, R_{d}, Rₑ, R_{f}, R_{g}, Rₕ and Rᵢ are independently selected from hydrogen, C₁-C₆ alkyl and C₁-C₆ haloalkyl;
to provide a compound of formula (IX) or a salt or solvate thereof.

Compounds of formula (I) and (Ill), and salts or solvates thereof, can be used as intermediates in the preparation of Zuranolone, and other related compounds of formula (IX). Therefore, in another aspect the invention is directed to a compound selected from:

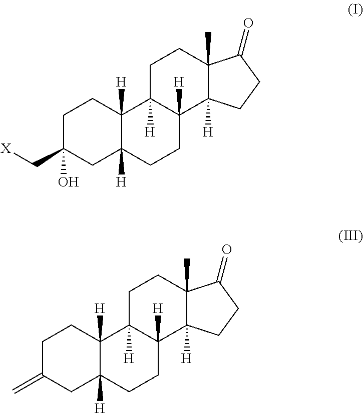

or a salt or solvate thereof, wherein X is selected from Cl, Br and I.

### DETAILED DESCRIPTION OF THE INVENTION

As used herein, the singular forms “a” “an” and “the” include plural reference unless the context clearly dictates otherwise.

The term “C₁-C₆ alkyl” refers to a linear or branched hydrocarbon chain radical consisting of carbon and hydrogen atoms, containing no unsaturation, having between 1 and 6, or between 1 and 3 (“C₁-C₃ alkyl”), carbon atoms and which is attached to the rest of the molecule through a single bond. Examples of alkyl groups include methyl, ethyl, n-propyl, i-propyl, n-butyl, i-butyl, t-butyl, n-pentyl, hexyl.

The term “C₃-C₇ cycloalkyl” refers to a saturated or partially saturated mono- or bicyclic aliphatic group having between 3 and 7, or between 3 and 6 (“C₃-C₆ cycloalkyl”) carbon atoms. Examples of cycloalkyl groups include cyclopropyl, cyclobutyl, cyclopentyl, cyclohexyl.

The term “C₁-C₆ alkoxyl” designates an alkyl group as defined above having between 1 and 6 carbon atoms, or between 1 and 3 carbon atoms (“C₁-C₃ alkoxyl”), linked to the rest of the molecule through oxygen. Examples of alkoxy include methoxy, ethoxy, isopropoxy, tertbutoxy.

The term “halogen” refers to bromine, chlorine, iodine or fluorine.

The term “C₁-C₆ haloalkyl” refers to an alkyl group as defined above wherein at least one of the hydrogen atoms has been replaced by a halogen atom such as, for example CF₃, CCl₃, CHF₂, CH₂F, CF₂CF₃.

The term “C₆-C₁₀ aryl” refers to an aromatic group having between 6 and 10, or 6 or 10 carbon atoms, comprising 1 or 2 aromatic nuclei. Examples of aryl groups include phenyl, naphthyl, indenyl, phenanthryl.

The term “(C₆-C₁₀)aryl(C₁-C₆)alkyl” refers to an aryl group as defined above which is attached to the rest of the molecule through an alkyl group as defined above. Examples of such groups include benzyl, phenylethyl, phenylpropyl, naphthylmethyl.

The term “5- to 10-membered heterocyclyl” refers to a saturated or partially unsaturated monocyclic or bicyclic system containing from 5 to 10, or from 5 to 7, ring atoms that consists of carbon atoms and from one to five, or even 1, 2 or 3, heteroatoms selected from the group consisting of nitrogen, oxygen, and sulphur.

The term “5- to 10-membered heteroaryl” refers to an aromatic monocyclic or bicyclic system containing from 5 to 10, or from 5 to 7, ring atoms that consists of carbon atoms and from one to five, or even 1, 2 or 3, heteroatoms selected from the group consisting of nitrogen, oxygen, and sulphur.

As understood in this technical area, there may be a certain degree of substitution in the aforementioned radicals. Therefore, there may be substitution in any of the groups of the present invention. The previous groups can be substituted in one or more available positions with one or more substituents, e.g. one, two or three substituents. Said substituents include, for example, C₁₋₆ alkyl, C₁₋₆ haloalkyl, C₃₋₇ cycloalkyl, (C₆-C₁₀)aryl(C₁-C₆)alkyl, C₆-C₁₀ aryl, 5- to 10-membered heterocyclyl, 5- to 10-membered heteroaryl, halogen, —CN, NO₂, —N(Rₐ)(R_{b}), —OR_{c}, —SR_{d}, —C(O)Rₑ, —C(O)OR_{f}, —C(O)N(R_{g})(Rₕ), —OC(O)Rᵢ; wherein Rₐ, R_{b}, R_{c}, R_{d}, Rₑ, R_{f}, R_{g}, Rₕ and Rᵢ are independently selected from hydrogen, C₁-C₆ alkyl, C₁₋₆ haloalkyl, (C₆-C₁₀)aryl(C₁-C₆)alkyl, C₆-C₁₀ aryl, 5- to 10-membered heterocyclyl and 5- to 10-membered heteroaryl.

The invention also provides “salts” of the compounds described herein. By way of illustration, said salts can be acid addition salts, base addition salts or metal salts, and can be synthesized from the parent compounds containing a basic or acid moiety by means of conventional chemical processes known by the persons skilled in the art. Such salts are generally prepared, for example, by reacting the free acid or base forms of said compounds with a stoichiometric amount of the suitable base or acid in water or in an organic solvent or in a mixture of the two. Non-aqueous media such as ether, ethyl acetate, ethanol, acetone, isopropanol or acetonitrile are generally preferred. Illustrative examples of acid addition salts include inorganic acid addition salts such as, for example, hydrochloride, hydrobromide, hydroiodide, sulfate, nitrate, phosphate, etc., organic acid addition salts such as, for example, acetate, maleate, fumarate, citrate, oxalate, succinate, tartrate, malate, mandelate, methanesulfonate, p-toluenesulfonate, camphorsulfonate, etc. Illustrative examples of base addition salts include inorganic base salts such as, for example, ammonium salts and organic base salts such as, for example, ethylenediamine, ethanolamine, N,N-dialkylenethanolamine, triethanolamine, glutamine, amino acid basic salts, etc. Illustrative examples of metal salts include, for example, sodium, potassium, calcium, magnesium, aluminium and lithium salts.

The term “solvate” according to this invention is to be understood as meaning any form of the compound which has another molecule (most likely a polar solvent) attached to it via non-covalent bonding. Examples of solvate include hydrates and alcoholates, e.g. methanolates. Solvation methods are generally known in the state of the art.

The term “organic solvent” includes for example cyclic and acyclic ethers (e.g. Et₂O, iPr₂O, tBu₂O, MeOtBu, 1,4-dioxane, 1,3-dioxolane, 1,2-dimethoxyethane, tetrahydrofuran, methyltetrahydrofuran), hydrocarbon solvents (e.g. pentane, hexane, heptane), halogenated solvents (e.g. dichloromethane, chloroform), aromatic solvents (e.g. toluene, xylene), ketones (e.g. acetone, butanone, pentanone, methyl ethyl ketone, ethyl isopropyl ketone), esters (e.g. EtOAc, iPrOAc, BuOAc), nitriles (e.g. acetonitrile, benzonitrile), amides (e.g. DMF, DMA, HMPA, NMP), alcohols (e.g. methanol, ethanol, propanol, isopropanol, sec-butanol, t-butanol), sulfoxides (DMSO) and mixtures thereof.

The term “aprotic organic solvent” means any organic solvent that does not yield a proton under the reaction conditions. Suitable examples include, but are not limited to, cyclic and acyclic ethers (e.g. Et₂O, iPr₂O, tBu₂O, MeOtBu, 1,4-dioxane, 1,3-dioxolane, 1,2-dimethoxyethane, tetrahydrofuran, methyltetrahydrofuran), hydrocarbon solvents (e.g. pentane, hexane, heptane), halogenated solvents (e.g. dichloromethane, chloroform), aromatic solvents (e.g. toluene, xylene), ketones (e.g. acetone, butanone, pentanone, methyl ethyl ketone, ethyl isopropyl ketone), esters (e.g. EtOAc, iPrOAc, BuOAc), nitriles (e.g. acetonitrile, benzonitrile), amides (e.g. DMF, DMA, HMPA, NMP), sulfoxides (DMSO) and mixtures thereof.

In an aspect, the invention is directed to a process for preparing a compound of formula (I) or a salt or solvate thereof

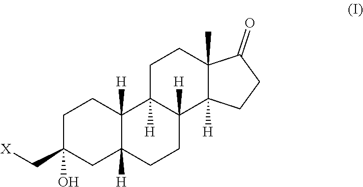

wherein X is selected from Cl, Br and I; which comprises
   (a) olefination of a compound of formula (II) or a salt or solvate thereof

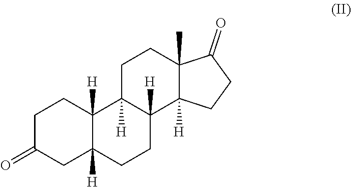

to provide a compound of formula (III) or a salt or solvate thereof

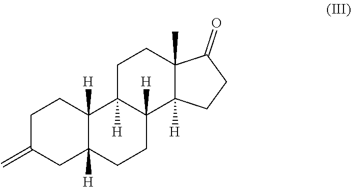

and
   (b) halohydrin formation reaction of the compound of formula (III), or a salt or solvate thereof, to provide the compound of formula (I), or a salt or solvate thereof.

In an embodiment, the process of the invention further comprises:

(c) conversion of the compound of formula (I), or a salt or solvate thereof, into a compound of formula (IV), or a salt or solvate thereof

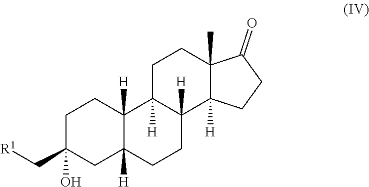

wherein R¹ is selected from H, F, C₁₋₆ alkyl, C₁₋₆ alkoxyl, C₁₋₆ haloalkyl, and N(R′)₂, wherein
each R′ is independently selected from H and C₁₋₆ alkyl.

Thus, in another aspect, the invention is directed to a process for preparing a compound of formula (IV), or a salt or solvate thereof, which comprises steps (a), (b) and (c) as defined herein.

In another embodiment, the process of the invention further comprises:

(c) conversion of the compound of formula (I), or a salt or solvate thereof, into a compound of formula (IV), or a salt or solvate thereof

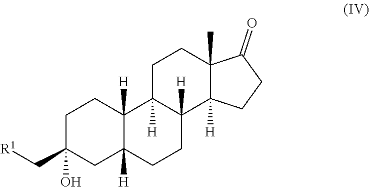

wherein R¹ is selected from H, F, C₁₋₆ alkyl, C₁₋₆ alkoxyl, C₁₋₆ haloalkyl, and N(R′)₂, wherein
each R′ is independently selected from H and C₁₋₆ alkyl; and
   (d) olefination of the compound of formula (IV), or a salt or solvate thereof, to provide a compound of formula (V), or a salt or solvate thereof

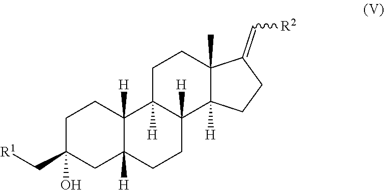

wherein
R¹ is selected from H, F, C₁₋₆ alkyl, C₁₋₆ alkoxyl, C₁₋₆ haloalkyl, and N(R′)₂, wherein each R′ is independently selected from H and C₁₋₆ alkyl; and
R² is selected from H and C₁₋₆ alkyl.

Thus, in another aspect, the invention is directed to a process for preparing a compound of formula (V), or a salt or solvate thereof, which comprises steps (a), (b), (c) and (d) as defined herein.

In another embodiment, the process of the invention further comprises:
(c) conversion of the compound of formula (I), or a salt or solvate thereof, into a compound of formula (IV), or a salt or solvate thereof

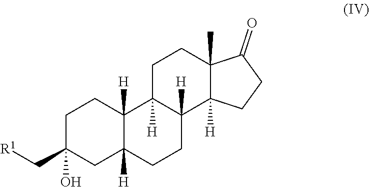

wherein R¹ is selected from H, F, C₁₋₆ alkyl, C₁ 6 alkoxyl, C₁₋₆ haloalkyl, and N(R′)₂, wherein each R′ is independently selected from H and C₁₋₆ alkyl;
   (d) olefination of the compound of formula (IV), or a salt or solvate thereof, to provide a compound of formula (Va), or a salt or solvate thereof

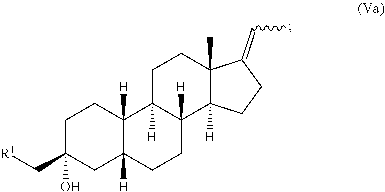

(e) hydroxylation of the compound of formula (Va), or a salt or solvate thereof, to provide a compound of formula (VI), or a salt or solvate thereof

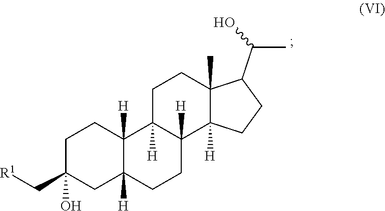

(f) oxidation of the compound of formula (VI), or a salt or solvate thereof, to provide a compound of formula (VII), or a salt or solvate thereof

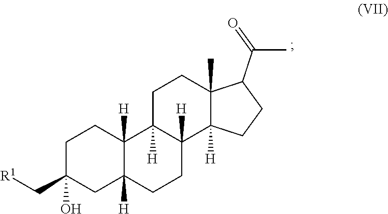

(g) halogenation of the compound of formula (VII), or a salt or solvate thereof, to provide a compound of formula (VIII), or a salt or solvate thereof

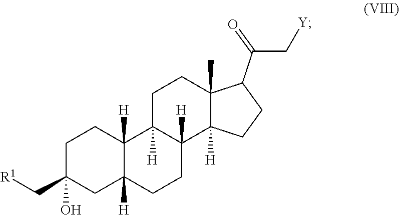

wherein Y is halogen; and
   (h) reaction of the compound of formula (VIII), or a salt or solvate thereof, with a 5- to 10-membered heterocycle or a 5- to 10-membered heteroaryl, wherein the 5- to 10-membered heterocycle and the 5- to 10-membered heteroaryl are unsubstituted or substituted with a substituent selected from the group consisting of C₁₋₆ alkyl, C₁₋₆ haloalkyl, halogen, —CN, NO₂, —N(Rₐ)(R_{b}), —OR_{c}, —SR_{d}, —C(O)Rₑ, —C(O)OR_{f}, —C(O)N(R_{g})(Rₕ) and —OC(O)Rᵢ; wherein Rₐ, R_{b}, R_{c}, R_{d}, Rₑ, R_{f}, R_{g}, Rₕ and Rᵢ are independently selected from hydrogen, C₁-C₆ alkyl and C₁-C₆ haloalkyl;
to provide a compound of formula (IX) or a salt or solvate thereof

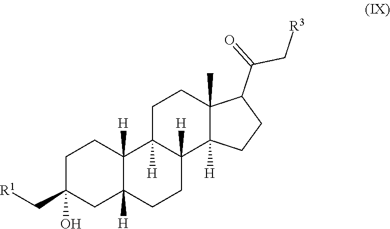

wherein R³ is selected from 5- to 10-membered heterocyclyl and 5- to 10-membered heteroaryl, wherein the 5- to 10-membered heterocyclyl and the 5- to 10-membered heteroaryl are unsubstituted or substituted with a substituent selected from the group consisting of C₁₋₆ alkyl, C₁₋₆ haloalkyl, halogen, —CN, NO₂, —N(Rₐ)(R_{b}), —OR_{c}, —SR_{d}, —C(O)Rₑ, —C(O)OR_{f}, —C(O)N(R_{g})(Rₕ) and —OC(O)Rᵢ; wherein Rₐ, R_{b}, R_{c}, R_{d}, Rₑ, R_{f}, R_{g}, Rₕ and Rᵢ are independently selected from hydrogen, C₁-C₆ alkyl and C₁-C₆ haloalkyl.

Thus, in another aspect, the invention is directed to a process for preparing a compound of formula (IX), or a salt or solvate thereof, which comprises steps (a), (b), (c), (d), (e), (f), (g) and (h) as defined herein.

In an embodiment, X is Br or I. In a further embodiment, X is Br.

In an embodiment, R¹ is selected from H, F, C₁₋₃ alkyl, C₁₋₃ alkoxyl, C₁₋₃ haloalkyl, and N(R′)₂, wherein each R′ is independently selected from H and C₁₋₃ alkyl.

In an embodiment, R¹ is selected from H, F, C₁₋₆ alkyl and C₁₋₆ alkoxyl. In a further embodiment, R¹ is selected from H, F, Me, Et, —OMe and —OEt. According to a particular embodiment, R¹ is H.

In an embodiment, R² is selected from H and C₁₋₃ alkyl. In a further embodiment, R² is selected from H and Me. According to a particular embodiment, R² is Me. According to another embodiment, R² is H.

In an embodiment, the 5- to 10-membered heterocyclyl and 5- to 10-membered heteroaryl in R³ comprises a N atom through which it is bound to the rest of the molecule. Thus, in an embodiment, R³ is a group of formula

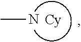

wherein Cy is selected from 5- to 10-membered heterocyclyl and 5- to 10-membered heteroaryl, wherein the 5- to 10-membered heterocyclyl and the 5- to 10-membered heteroaryl are unsubstituted or substituted with a substituent selected from the group consisting of C₁₋₆ alkyl, C₁₋₆ haloalkyl, halogen, —CN, NO₂, —N(Rₐ)(R_{b}), —OR_{c}, —SR_{d}, —C(O)Rₑ, —C(O)OR_{f}, —C(O)N(R_{g})(Rₕ) and —OC(O)Rᵢ; wherein Rₐ, R_{b}, R_{c}, R_{d}, Rₑ, R_{f}, R_{g}, Rₕ and Rᵢ are independently selected from hydrogen, C₁-C₆ alkyl and C₁-C₆ haloalkyl.

Therefore, in a particular embodiment, step (h) comprises reaction of the compound of formula (VIII), or a salt or solvate thereof, with a compound of formula

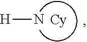

wherein Cy is as defined herein.

In an embodiment, Rₐ, R_{b}, R_{c}, R_{d}, Rₑ, R_{f}, R_{g}, Rₕ and Rᵢ are independently selected from hydrogen, C₁-C₃ alkyl and C₁-C₃ haloalkyl.

In a particular embodiment, R³ or Cy are selected from a 5- to 10-membered heterocyclyl and 5- to 10-membered heteroaryl, wherein the 5- to 10-membered heterocyclyl and the 5- to 10-membered heteroaryl are unsubstituted or substituted with a substituent selected from the group consisting of C₁₋₃ alkyl, C₁₋₃ haloalkyl, halogen, —CN, NO₂, —N(Rₐ)(R_{b}), —OR_{c}, —SRₐ, —C(O)Rₑ, —C(O)OR_{f}, —C(O)N(R_{g})(Rₕ) and —OC(O)Rᵢ; wherein Rₐ, R_{b}, R_{c}, R_{d}, Rₑ, R_{f}, R_{g}, Rₕ and Rᵢ are independently selected from hydrogen, C₁-C₃ alkyl and C₁-C₃ haloalkyl.

In an embodiment, R³ or Cy are selected from a 5- or 6-membered heterocyclyl and 5- or 6-membered heteroaryl, wherein the 5- or 6-membered heterocyclyl and the 5- or 6-membered heteroaryl are unsubstituted or substituted with a substituent selected from the group consisting of C₁₋₆ alkyl, C₁₋₆ haloalkyl, halogen, —CN, NO₂, —N(Rₐ)(R_{b}), —OR_{c}, —SRₐ, —C(O)Rₑ, —C(O)OR_{f}, —C(O)N(R_{g})(Rₕ) and —OC(O)Rᵢ; wherein Rₐ, R_{b}, R_{c}, R_{d}, Rₑ, R_{f}, R_{g}, Rₕ and Rᵢ are independently selected from hydrogen, C₁-C₆ alkyl and C₁-C₆ haloalkyl.

In a further embodiment, R³ or Cy are selected from a 5- or 6-membered heterocyclyl and 5- or 6-membered heteroaryl, wherein the 5- or 6-membered heterocyclyl and the 5- or 6-membered heteroaryl are unsubstituted or substituted with a substituent selected from the group consisting of C₁₋₃ alkyl, C₁₋₃ haloalkyl, halogen, —CN, NO₂, —N(Rₐ)(R_{b}), —OR_{c}, —SR_{d}, —C(O)Rₑ, —C(O)OR_{f}, —C(O)N(R_{g})(Rₕ) and —OC(O)Rᵢ; wherein Rₐ, R_{b}, R_{c}, R_{d}, Rₑ, R_{f}, R_{g}, Rₕ and Rᵢ are independently selected from hydrogen, C₁-C₃ alkyl and C₁-C₃ haloalkyl.

According to an embodiment, R³ or Cy are selected from pyrrolidine, pyperidine, piperazine, morpholine, pyrrolepyrazole, imidazole, 1,2,3-triazole, 1,2,4-triazole, tetrazole, indole, isoindole, benzimidazole, indazole, benzotriazole, pyrazolo[3,4-b]pyridine, pyrazolo[3,4-c]pyridine, pyrazolo[4,3-b]pyridine, pyrazolo[4,3-c]pyridine, imidazo[4,5-b]pyridine, imidazo[4,5-c]pyridine, pyrazolo[3,4-d]pyrimidine, pyrazolo[4,3-d]pyrimidine, purine, pyrazolo[3,4-b]pyrazine, imidazo[4,5-b]pyrazine, benzotriazole, 1,2,3-triazolo[4,5-b]pyridine, 1,2,3-triazolo[4,5-c]pyridine, 1,2,3-triazolo[4,5-d]pyrimidine, 1,2,3-triazolo[4,5-b]pyrazine, wherein said groups can be unsubstituted or substituted with C₁₋₆ alkyl, C₁₋₆ haloalkyl, halogen, —CN, NO₂, —N(Rₐ)(R_{b}), —OR_{c}, —SR_{d}, —C(O)Rₑ, —C(O)OR_{f}, —C(O)N(R_{g})(Rₕ) or —OC(O)Rᵢ; wherein Rₐ, R_{b}, R_{c}, R_{d}, Rₑ, R_{f}, R_{g}, Rₕ and Rᵢ are independently selected from hydrogen, C₁-C₆ alkyl and C₁-C₆ haloalkyl.

In an embodiment, the substituent on the R³ or Cy group can be selected from C₁₋₃ alkyl, C₁₋₃ haloalkyl, halogen, —CN, NO₂, —N(Rₐ)(R_{b}), —OR_{c}, —SR_{d}, —C(O)Rₑ, —C(O)OR_{f}, —C(O)N(R_{g})(Rₕ) and —OC(O)Rᵢ; wherein Rₐ, R_{b}, R_{c}, R_{d}, Rₑ, R_{f}, R_{g}, Rₕ and Rᵢ are independently selected from hydrogen, C₁-C₃ alkyl and C₁-C₃ haloalkyl. In a further embodiment, the substituent on the R³ group can be selected from Me, Et, CF₃, F, Cl, —CN, —NH₂, OMe, OEt, SMe, OCF₃, —COMe, —COOH, —COOMe, —CONH₂, —CONHMe and —CONMe₂.

In a particular embodiment, R³ or Cy are

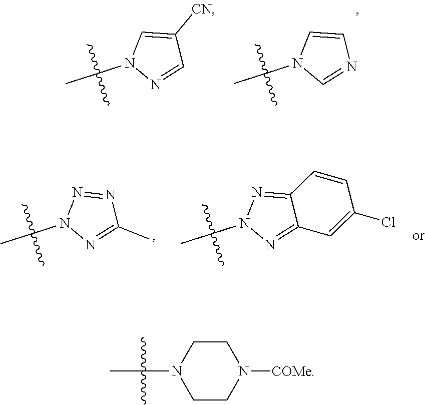

In an embodiment, R³ or Cy are

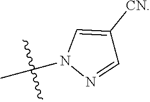 [SMILES: *n1cc(C#N)cn1]

In a particular embodiment, the compound of formula (IX) is selected from:

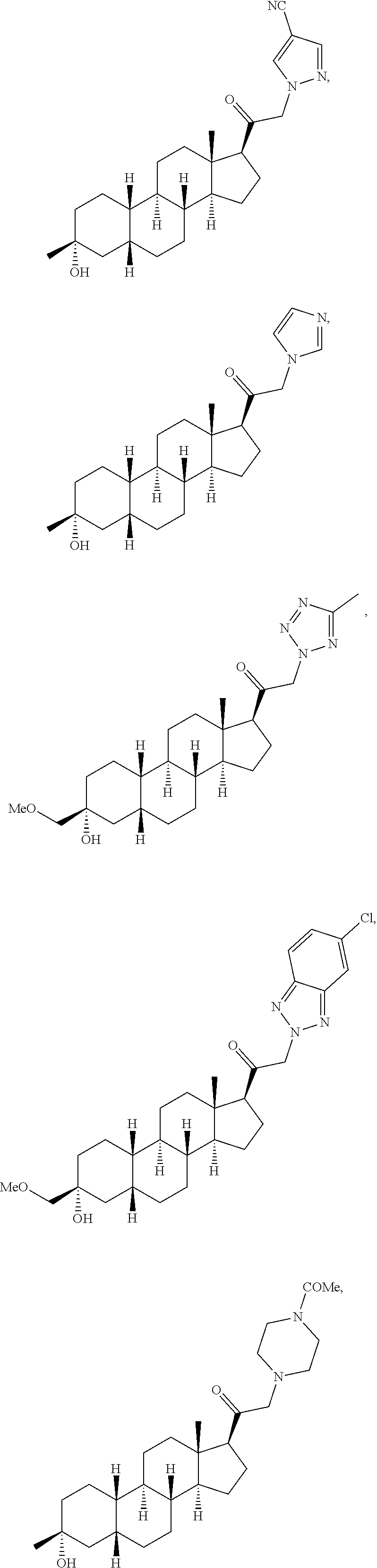

or a salt or solvate thereof.

In an embodiment, the compound of formula (IX) is zuranolone, or a salt or solvate thereof.

### (a) Olefination of a Compound of Formula (II)

The compound of formula (III), or a salt or solvate thereof, is obtained by olefination of a compound of formula (II), or a salt or solvate thereof.

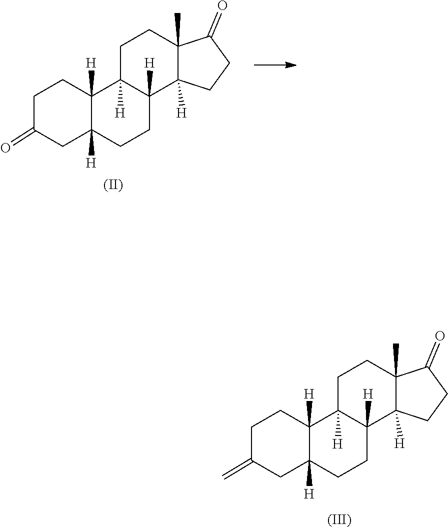

Olefination reactions of ketones, such as Wittig reaction, and suitable reaction conditions are known in the art.

In an embodiment, olefination is performed by reaction with a compound of formula (X)

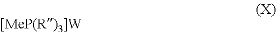

wherein
   W is halogen, such as Br; and
   each R″ is independently selected from C₆-C₁₀ aryl, such as phenyl;
in the presence of a base.

In an embodiment, the compound of formula (X) is methyltriphenylphosphonium bromide.

Suitable bases include organolithium bases, alkali metal hydrides and alkali metal C₁₋₆ alkoxides, such as e.g. nBuLi, tBuLi, sBuLi, MeLi, PhLi, HMDSLi, LDA, NaH, NaOtBu, KOtBu, NaOMe, NaOEt. In a particular embodiment, the base is an alkali metal C₁₋₆ alkoxide, such as KOtBu.

Preferably, the reaction is carried out in the presence of an organic solvent, such as for example a cyclic or acyclic ether (e.g. Et₂O, iPr₂O, tBu₂O, MeOtBu, 1,4-dioxane, 1,3-dioxolane, 1,2-dimethoxyethane, tetrahydrofuran, methyltetrahydrofuran), a hydrocarbon solvent (e.g. pentane, hexane, heptane), a halogenated solvent (e.g. dichloromethane, chloroform), an aromatic solvent (e.g. toluene, xylene), an amide (e.g. DMF, DMA) or mixtures thereof. In a particular embodiment, the solvent is a cyclic or acyclic ether, such as THF.

In a particular embodiment, in the olefination reaction in step (a), the compound of formula (X) is methyltriphenylphosphonium bromide, the base is KOtBu and the organic solvent is THF.

In an embodiment, the reaction is carried out at a temperature between −20° C. and 50° C., or between 0° C. and 20° C.

The compound of formula (X) and/or the base is preferably used in an amount of 1.0-1.5 molar equivalents, or 1.0-1.2 molar equivalents, with respect to the compound of formula (II), or a salt or solvate thereof.

Also, in a particular embodiment, a mixture of the compound of formula (X) and the base is first formed in an organic solvent and the resulting mixture is added over a mixture of the compound of formula (II), or a salt or solvate thereof, and an organic solvent.

### (b) Halohydrin Formation Reaction

The compound of formula (I), or a salt or solvate thereof, is obtained by halohydrin formation reaction of a compound of formula (III), or a salt or solvate thereof.

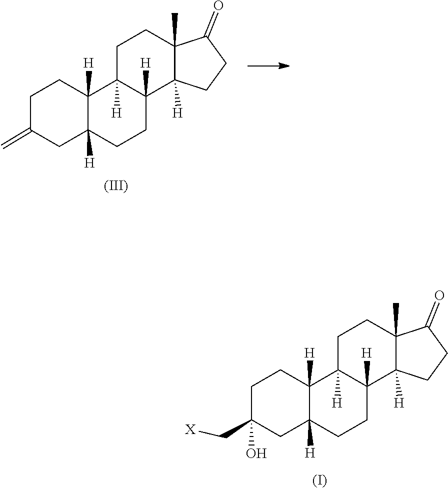

In this reaction, the alkene is converted into a halohydrin. The inventors have observed that this reaction proceeds in a regioselective and in a stereoselective manner, to yield the halohydrin on which the halogen atom is bonded to the terminal carbon atom and that has a beta-halomethyl alpha-hydroxy configuration as the major or only product.

This reaction can be performed in the presence of a halohydrin forming reagent. Typical halohydrin forming reagents include HOCl, HOBr, HOI, and combinations of a halogenating agent (e.g. N-chlorosuccinimide, N-bromosuccinimide, N-iodosuccinimide, C₂, Br₂, I₂, DCDMH, DBDMH or DIDMH) and water.

In an embodiment, X is Br, and the halohydrin forming reagent is selected from HOBr, N-bromosuccinimide and water, Br₂ and water, and DBDMH and water. In a particular embodiment, it is selected from N-bromosuccinimide and water, and DBDMH and water, or even it is N-bromosuccinimide and water.

In another embodiment, X is I, and the halohydrin forming reagent is selected from HOI, N-iodosuccinimide and water, I₂ and water, and DIDMH and water. In a particular embodiment, it is selected from N-iodosuccinimide and water, and DIDMH and water, or even it is N-iodosuccinimide and water.

The halohydrin forming reagent may be used in an amount of 1-8 molar equivalents, or 1-3 molar equivalents, with respect to the compound of formula (III), or a salt or solvate thereof. When the halohydrin forming reagent is a combination of a halogenating agent and water, the halogenating agent may be used in an amount of 1-8 molar equivalents, or 1-3 molar equivalents, with respect to the compound of formula (III), or a salt or solvate thereof. In this case, the water can be used in an excess amount, e.g. it can be used in an amount of 1-100 molar equivalents, or 5-60 molar equivalents, with respect to the compound of formula (III), or a salt or solvate thereof.

In an embodiment, the reaction is performed in the presence of an acid, for instance a strong acid (e.g. pKa<1), such as HClO₄, MeSO₃H, p-ToISO₃H, CF₃SO₃H, PhSO₃H, HCl, HBr, HI, H₂SO₄, HNO₃, CF₃CO₂H. In an embodiment, the reaction is performed in the presence of HClO₄.

The reaction may be performed in the presence of an organic solvent, such as an aprotic organic solvent; water, or a mixture thereof. In an embodiment, the reaction is carried out in the presence of acetone or a mixture of acetone and water.

In a particular embodiment, the reaction in step (b) is performed in the presence of N-bromosuccinimide or N-iodosuccinimide; water; HClO₄; and an organic acid, such as acetone.

In an embodiment, the reaction is carried out at a temperature between −30° C. and 50° C., or between −20° C. and 20° C.

In another embodiment, the reaction is carried out at a temperature between −20° C. and 0° C. In a further embodiment, the reaction is carried out at a temperature between −15° C. and −10° C.

The inventors have observed, that halohydrin formation reaction of compounds of formula (III) results in compounds of formula (I) with high stereoselectivity. In an embodiment, the compound of formula (I) is obtained in an amount of at least 75% with respect to the sum of the two stereoisomers at position 3 of the steroid. That is, the alpha/beta ratio of the hydroxyl group is ≥75/≤25. In a particular embodiment, said amount is at least 80% (alpha/beta ratio of the hydroxyl group is ≥80/≤20) or even at least 85% (alpha/beta ratio of the hydroxyl group is ≥85/≤15). In a further embodiment, said amount is at least 90% (alpha/beta ratio of the hydroxyl group is ≥90/≤10). The ratio of stereoisomers can be determined by HPLC.

The inventors have observed that even when some amount of the undesired stereoisomer (beta hydroxyl isomer at position 3) is formed in the halohydrin formation reaction, purification of the compound of formula (I) allows to easily remove or significantly reduce the low amounts of the undesired stereoisomer.

In a particular embodiment, after step b), the compound of formula (I) is purified, for example by column chromatography or by recrystallization. In an embodiment, recrystallization can be performed in an organic solvent or a mixture of organic solvents, such as Et₂O, iPr₂O, tBu₂O, MeOtBu, 1,4-dioxane, 1,3-dioxolane, 1,2-dimethoxyethane, tetrahydrofuran, methyltetrahydrofuran, pentane, hexane, heptane, dichloromethane, chloroform, toluene, xylene, acetone, butanone, pentanone, methyl ethyl ketone, ethyl isopropyl ketone, EtOAc, iPrOAc, BuOAc, acetonitrile, methanol, ethanol, propanol, isopropanol, sec-butanol, t-butanol, DMSO and mixtures thereof. In an embodiment, recrystallization can be performed in a mixture of iPr₂O and heptane.

(c) Conversion of a Compound of Formula (I) into a Compound of Formula (IV)

The compound of formula (IV), or a salt or solvate thereof, can be obtained from a compound of formula (I), or a salt or solvate thereof.

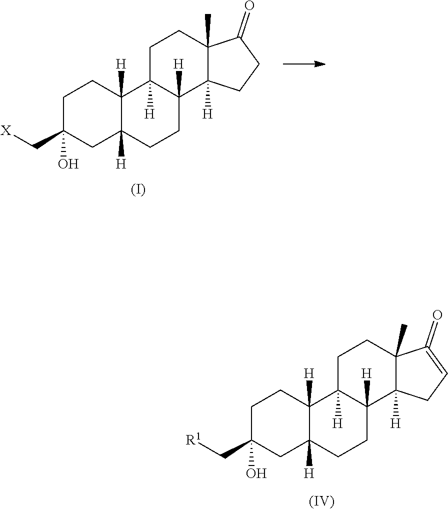

In a particular embodiment, R¹ in the compound of formula (IV), or a salt or solvate thereof, is H. In said embodiment, step (c) comprises dehalogenation of the compound of formula (I), or a salt or solvate thereof, to provide a compound of formula (IV), or a salt or solvate thereof, wherein R¹ is H.

Dehalogenation reactions and suitable reaction conditions are known in the art. In an embodiment, this dehalogenation reaction is performed in the presence of a tin hydride and a radical initiator.

Tin hydrides include, for example, tributyltin hydride, triphenyltin hydride, trimethyltin hydride, dimethyltin dihydride, dioctyltin dihydride, diisobutyltin dihydride, and tridecyltin hydride. In an embodiment, the tin hydride is selected from tributyltin hydride, triphenyltin hydride, or even it is tributyltin hydride.

The tin hydride may be used in an amount of 1-8 molar equivalents, or 1.2-3 molar equivalents, with respect to the compound of formula (I), or a salt or solvate thereof.

Radical initiators are well known in the art and include, among others, alkyl and aryl peroxides, alkyl and aryl hydroperoxides, acyl peroxides, peroxyesters, persulfates, perborates, percarbonates and azo compounds. Some specific examples include hydrogen peroxide, dibenzoyl peroxide, didecanoyl peroxide, dilauroyl peroxide, t-butyl hydroperoxide, benzoyl peroxide, di-t-butyl peroxide, di(3,5,5-trimethylhexanoyl)peroxide, diisobutyryl peroxide, t-butylperoxy diethyl acetate, t-butyl peroctoate, t-butyl peroxy isobutyrate, t-butyl peroxy 3,5,5-trimethyl hexanoate, t-butyl perbenzoate, t-butyl peroxy pivalate, t-butyl peroxy-2-ethyl hexanoate, tert-amyl peroxy-2-ethylhexanoate, 1,1,3,3-tetramethylbutyl peroxy-2-ethylhexanoate and cumene hydroperoxide, AIBN and 2,2′-azobis-(2-methylbutyronitrile). In an embodiment, the radical initiator is AIBN.

The radical initiator may be used in a catalytic amount, such as 0.01-0.6 molar equivalents with respect to the compound of formula (I), or a salt or solvate thereof.

The dehalogenation reaction may be performed in the presence of an organic solvent, such as an aprotic organic solvent. In a particular embodiment, the solvent is a cyclic or acyclic ether, such as THF.

In a particular embodiment, the dehalogenation reaction is performed in the presence of a tin hydride, a radical initiator and an organic solvent.

In an embodiment, the dehalogenation reaction is carried out at a temperature between 20° C. and 150° C., or between 40° C. and 100° C.

In another embodiment, R¹ in the compound of formula (IV), or a salt or solvate thereof, is F. In said embodiment, step (c) comprises treatment of the compound of formula (I), or a salt or solvate thereof, with a base and then with a fluoride source to provide a compound of formula (IV), or a salt or solvate thereof, wherein R¹ is F.

In the fluorination reaction, the compound of formula (IV), or a salt or solvate thereof, is first treated with a base. Suitable bases include inorganic and organic bases, such as an alkali metal carbonate or bicarbonate (e.g. Na₂CO₃, K₂CO₃, Cs₂CO₃, Li₂CO₃, NaHCO₃, KHCO₃, CsHCO₃, LiHCO₃), an alkali metal phosphate (e.g. Na₃PO₄, K₃PO₄, Na₂HPO₄, K₂HPO₄, NaH₂PO₄, KH₂PO₄), an alkali metal alkoxide (e.g. NaOMe, KOMe, NaOEt, KOEt, NaOtBu, KOtBu), an alkali metal hydroxide (e.g. NaOH, KOH, LiOH, CsOH), an aliphatic or aromatic amine (e.g. Me₂NH, Et₂NH, iPr₂NH, Bu₂NH, Me₃N, Et₃N, Bu₃N, iPr₂EtN, N-methylmorpholine, pyridine, DMAP, aniline, N,N-dimethylaniline). In an embodiment, the base is an inorganic base, such as an alkali metal carbonate, bicarbonate or phosphate. In an embodiment, the base is K₂CO₃.

After treatment with a base, the fluorination reaction comprises treatment of the resulting compound with a fluoride source. Fluoride sources are well-known in the art and include, for example, KF, tetramethylammonium fluoride, tetrabutylammonium fluoride, ammonium fluoride, ammonium bifluoride, ammonium borofluoride, and fluoroboric acid. In an embodiment, the fluoride source is tetrabutylammonium fluoride.

The base and the fluoride source may be used in an amount of 1.5 to 50 molar equivalents, or 5-40 molar equivalents, with respect to the compound of formula (I), or a salt or solvate thereof.

The fluorination reaction may be performed in the presence of an organic solvent, such as an aprotic organic solvent. In a particular embodiment, the solvent is a cyclic or acyclic ether, such as THF, or an aromatic solvent, such as toluene.

In an embodiment, the fluorination reaction is carried out at a temperature between 20° C. and 150° C., such as between 60° C. and 120° C.

In another embodiment, R¹ in the compound of formula (IV), or a salt or solvate thereof, is C₁₋₆ alkyl. In said embodiment, step (c) comprises reaction of the compound of formula (I), or a salt or solvate thereof, with a compound of formula (C₁₋₆ alkyl)MgZ, wherein Z is selected from Cl, Br, I, or with a compound of formula (C₁₋₆ alkyl)₂CuLi, to provide a compound of formula (IV), or a salt or solvate thereof, wherein R¹ is C₁₋₆ alkyl.

The compound of formula (C₁₋₆ alkyl)MgZ or (C₁₋₆ alkyl)₂CuLi may be used in an amount of 1.5 to 10 molar equivalents, or 2-6 molar equivalents, with respect to the compound of formula (I), or a salt or solvate thereof.

In an embodiment, the C₁₋₆ alkyl is a C₁₋₃ alkyl, such a Me or Et.

The reaction with a compound of formula (C₁₋₆ alkyl)MgZ or (C₁₋₆ alkyl)₂CuLi may be performed in the presence of an organic solvent, such as an aprotic organic solvent. In a particular embodiment, the solvent is a cyclic or acyclic ether, such as THF.

In an embodiment, the reaction with a compound of formula (C₁₋₆ alkyl)MgZ or (C₁₋₆ alkyl)₂CuLi is carried out at a temperature between −80° C. and 20° C., or between −80° C. and 0° C. or even between −80° C. and −40° C.

In another embodiment, R¹ in the compound of formula (IV), or a salt or solvate thereof, is C₁₋₆ alkoxyl. In said embodiment, step (c) comprises reaction of the compound of formula (I), or a salt or solvate thereof, with a compound of formula (C₁₋₆ alkoxyl)M, wherein M is selected from Na and K, to provide a compound of formula (IV), or a salt or solvate thereof, wherein R¹ is C₁₋₆ alkoxyl.

The compound of formula (C₁₋₆ alkoxyl)M may be used in an amount of 1.5 to 10 molar equivalents, or 2-8 molar equivalents, with respect to the compound of formula (I), or a salt or solvate thereof.

In an embodiment, the C₁₋₆ alkoxyl is a C₁₋₃ alkoxyl, such a —OMe or —OEt.

The reaction with a compound of formula (C₁₋₆ alkoxyl)M may be performed in the presence of an organic solvent, such as an alcohol (e.g. MeOH, EtOH, nPrOH, iPrOH, sBuOH, tBuOH).

In an embodiment, the reaction with a compound of formula (C₁₋₆ alkoxyl)M is carried out at a temperature between 20° C. and 150° C., such as between 50° C. and 120° C.

In another embodiment, R¹ in the compound of formula (IV), or a salt or solvate thereof, is C₁₋₆ haloalkyl. In said embodiment, step (c) comprises reaction of the compound of formula (I), or a salt or solvate thereof, with a compound of formula (C₁₋₆ haloalkyl)MgZ, wherein Z is selected from Cl, Br, I, or with a compound of formula (C₁₋₆ haloalkyl)₂CuLi, to provide a compound of formula (IV), or a salt or solvate thereof, wherein R¹ is C₁₋₆ haloalkyl.

Preferably, the haloalkyl is a fluroalkyl, i.e. an alkyl group wherein at least one of the H atoms has been replaced by a F atom.

The compound of formula (C₁₋₆ haloalkyl)MgZ or (C₁₋₆ haloalkyl)₂CuLi may be used in an amount of 1.5 to 10 molar equivalents, or 2-6 molar equivalents, with respect to the compound of formula (I), or a salt or solvate thereof.

In an embodiment, the C₁₋₆ haloalkyl is a C₁₋₃ haloalkyl, such a CF₃, CHF₂, CH₂F or CF₂CF₃.

The reaction with a compound of formula (C₁₋₆ haloalkyl)MgZ or (C₁₋₆ haloalkyl)₂CuLi may be performed in the presence of an organic solvent, such as an aprotic organic solvent. In a particular embodiment, the solvent is a cyclic or acyclic ether, such as THF.

In an embodiment, the reaction with a compound of formula (C₁₋₆ haloalkyl)MgZ or C₁₋₆ haloalkyl)₂CuLi is carried out at a temperature between −80° C. and 20° C., or between −80° C. and 0° C. or even between −80° C. and −40° C.

In another embodiment, R¹ in the compound of formula (IV), or a salt or solvate thereof, is —N(R′)₂, wherein each R′ is independently selected from H and C₁₋₆ alkyl. In said embodiment, step (c) comprises reaction of the compound of formula (I), or a salt or solvate thereof, with a compound of formula HN(R′)₂, to provide a compound of formula (IV), or a salt or solvate thereof, wherein R¹ is —N(R′)₂, wherein each R′ is independently selected from H and C₁₋₆ alkyl.

In an embodiment, each R′ is independently selected from H and C₁₋₃ alkyl, such as H, Me or Et.

The compound of formula HN(R′)₂ may be used in an amount of 1 to 10 molar equivalents, or 1-6 molar equivalents, with respect to the compound of formula (I), or a salt or solvate thereof.

The reaction with a compound of formula HN(R′)₂ may be performed in the presence of an organic solvent, such as an aprotic organic solvent.

In an embodiment, the reaction with a compound of formula (HN(R′)₂ is carried out at a temperature between 20° C. and 120° C.

### (d) Olefination of a Compound of Formula (IV)

The compound of formula (V), or a salt or solvate thereof, may be obtained by olefination of a compound of formula (IV), or a salt or solvate thereof.

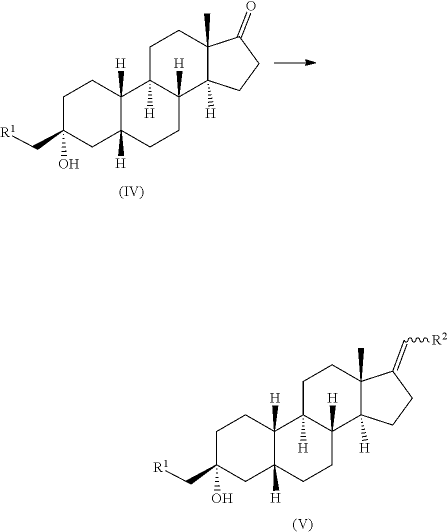

In a particular embodiment, R² is Me and so the compound of formula (V) has the formula (Va) as defined herein.

Olefination reactions of ketones, such as Wittig reaction, and suitable reaction conditions are known in the art.

In an embodiment, olefination is performed by reaction with a compound of formula

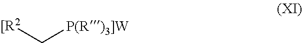

wherein
   W is halogen, such as Br,
   R² is selected from H and C₁₋₆ alkyl, and
   each R″′ is independently selected from C₆-C₁₀ aryl, such as phenyl;
in the presence of a base.

In an embodiment, R² is selected from H and C₁₋₃ alkyl, such as H, Me or Et. In a further embodiment, R² is selected from H and Me.

In an embodiment, the compound of formula (XI) is methyltriphenylphosphonium bromide or ethyltriphenylphosphonium bromide.

Suitable bases include organolithium bases, alkali metal hydrides and alkali metal C₁₋₆ alkoxide, such as e.g. nBuLi, tBuLi, sBuLi, MeLi, PhLi, HMDSLi, LDA, NaH, NaOtBu, KOtBu, NaOMe, NaOEt. In a particular embodiment, the base is an alkali metal C₁₋₆ alkoxide, such as KOtBu.

In the olefination reaction, the compound of formula (XI) and/or the base may be used in an amount of 1-10 molar equivalents, or 1-6 molar equivalents, with respect to the compound of formula (IV), or a salt or solvate thereof.

In an embodiment, the olefination reaction is carried out in the presence of an organic solvent, such as for example a cyclic or acyclic ether (e.g. Et₂O, iPr₂O, tBu₂O, MeOtBu, 1,4-dioxane, 1,3-dioxolane, 1,2-dimethoxyethane, tetrahydrofuran, methyltetrahydrofuran), a hydrocarbon solvent (e.g. pentane, hexane, heptane), a halogenated solvent (e.g. dichloromethane, chloroform), an aromatic solvent (e.g. toluene, xylene), an amide (e.g. DMF, DMA) or mixtures thereof. In a particular embodiment, the solvent is a cyclic or acyclic ether, such as THF.

In a particular embodiment, in the olefination reaction in step (d), the compound of formula (XI) is methyltriphenylphosphonium bromide or ethyltriphenylphosphonium bromide, the base is KOtBu and the organic solvent is THF.

In an embodiment, the reaction is carried out at a temperature between −20° C. and 60° C., or between 0° C. and 40° C.

Conversion of Compounds of Formula (IV) and (V) into Other Active Compounds

Compounds of formula (IV) and (V) have been disclosed in the prior art as key intermediates in the manufacture of compounds with biological activity, such as those disclosed, for instance, in WO2013/156181, WO2014/169832, WO2014/169833, WO2014/169836, WO2015/180679 or WO2020/118060, including zuranolone.

Therefore, the process of the present invention provides a very efficient method for the manufacture of very versatile key intermediates.

In an embodiment, the invention further comprises converting the compound of formula (V), or a salt or solvate thereof, into a compound of formula (IX), or a salt or solvate thereof. Methods for said conversion are known in the art (e.g. in WO2013/156181, WO2014/169832, WO2014/169833, WO2014/169836, WO2015/180679).

In a particular embodiment, the invention further comprises converting the compound of formula (Va), or a salt or solvate thereof, into a compound of formula (IX), or a salt or solvate thereof, by a process comprising:

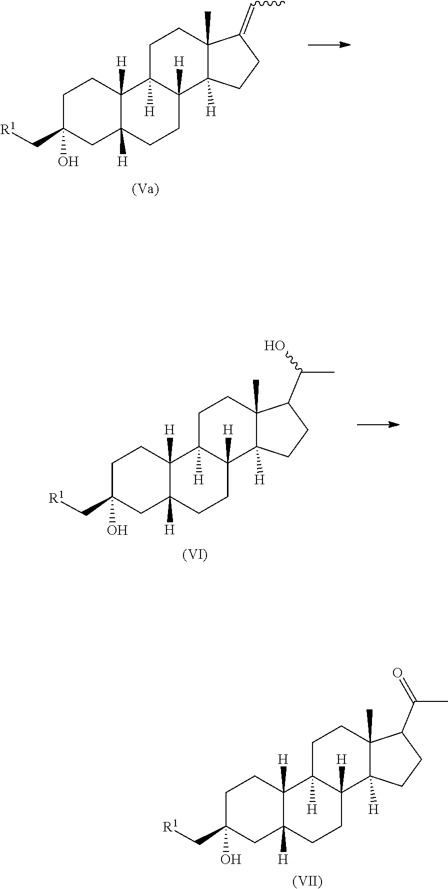

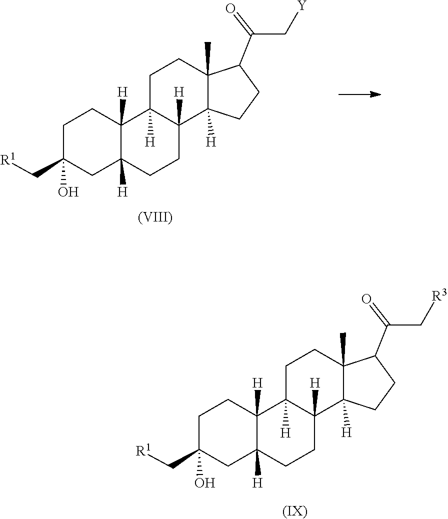

(e) hydroxylation of the compound of formula (Va), or a salt or solvate thereof, to provide a compound of formula (VI), or a salt or solvate thereof;
(f) oxidation of the compound of formula (VI), or a salt or solvate thereof, to provide a compound of formula (VII), or a salt or solvate thereof;
(g) halogenation of the compound of formula (VII), or a salt or solvate thereof, to provide a compound of formula (VIII), or a salt or solvate thereof; and
(h) reaction of the compound of formula (VIII), or a salt or solvate thereof, with a 5- to 10-membered heterocycle or a 5- to 10-membered heteroaryl, wherein the 5- to 10-membered heterocycle and the 5- to 10-membered heteroaryl are unsubstituted or substituted with C₁₋₆ alkyl, C₁₋₆ haloalkyl, halogen, —CN, NO₂, —N(Rₐ)(R_{b}), —OR_{c}, —SR_{d}, —C(O)Rₑ, —C(O)OR_{f}, —C(O)N(R_{g})(Rₕ), —OC(O)Rᵢ; wherein Rₐ, R_{b}, R_{c}, R_{d}, Rₑ, R_{f}, R_{g}, Rₕ and Rᵢ are independently selected from hydrogen, C₁-C₆ alkyl and C₁-C₆ haloalkyl; to provide a compound of formula (IX) or a salt or solvate thereof;
wherein
Y is halogen,
R¹ is selected from H, F, C₁₋₃ alkyl, C₁₋₆ alkoxyl, C₁₋₆ haloalkyl, and N(R′)₂, wherein each R′ is independently selected from H and C₁₋₆ alkyl, and
R³ is selected from 5- to 10-membered heterocyclyl and 5- to 10-membered heteroaryl, wherein the 5- to 10-membered heterocyclyl and the 5- to 10-membered heteroaryl are unsubstituted or substituted with a substituent selected from the group consisting of C₁₋₆ alkyl, C₁₋₆ haloalkyl, halogen, —CN, NO₂, —N(Rₐ)(R_{b}), —OR_{c}, —SR_{d}, —C(O)Rₑ, —C(O)OR_{f}, —C(O)N(R_{g})(Rₕ) and —OC(O)R; wherein Rₐ, R_{b}, R_{c}, R_{d}, Rₑ, R_{f}, R_{g}, Rₕ and Rᵢ are independently selected from hydrogen, C₁-C₆ alkyl and C₁-C₆ haloalkyl.

In an embodiment, Y is Br.

In an embodiment, R¹ is selected from H, F, C₁₋₃ alkyl and C₁₋₃ alkoxyl, such as H, F, Me, Et, —OMe and —OEt.

In a particular embodiment, R¹ is H and R³ is 4-cyanopyrazole.

In a further embodiment, the compound of formula (IX) is zuranolone or a salt or solvate thereof.

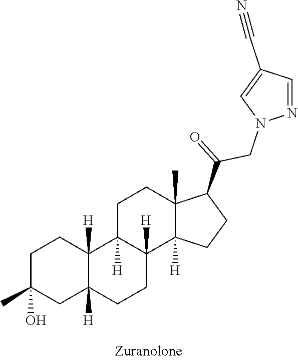

Methods and conditions for steps (e), (f), (g) and (h) are disclosed in the prior art (e.g. in WO2013/156181, WO2014/169832, WO2014/169833, WO2014/169836, WO2015/180679). In particular embodiments, said steps may be performed as follows.

In an embodiment, hydroxylation in step (e) may be performed by hydroboration-oxidation, such as treatment of the compound of formula (Va), or a salt or solvate thereof, with a borane followed by treatment with an oxidizing agent.

Hydroboration may be performed in the presence of a borane selected from BH₃, BH₃—SMe₂, BH₃-THF, BH₃-Et₂O, 9BBN, catecholborane or disiamilborane.

Oxidation of the resulting borane may be performed in the presence of an oxidizing agent, such as sodium perborate, hydrogen peroxide or sodium hypochlorite.

The hydroxylation in step (e) may be performed in presence of an organic solvent and optionally water. In an embodiment, the organic solvent is a cyclic or acyclic ether, such as THF.

In a particular embodiment, hydroxylation in step (e) is performed by treatment of the compound of formula (Va), or a salt or solvate thereof, with a borane (e.g. BH₃, BH₃—SMe₂, BH₃-THF, BH₃-Et₂O, 9BBN) in an organic solvent followed by treatment with sodium perborate, or with hydrogen peroxide and a base (e.g. NaOH).

In an embodiment, hydroxylation in step (e) is carried out at a temperature between −20° C. and 60° C., or between 0° C. and 40° C.

In an embodiment, oxidation in step (f) may be performed by treatment of the compound of formula (VI), or a salt or solvate thereof, with an oxidizing agent.

Suitable oxidizing agents include, among others, IBX, Dess-Martin periodinane (DMP), pyridinium chlorochromate (PCC), pyridinium dichromate (PDC), K₂Cr₂O₇, KMnO₄, MnO₂, CrO₃, RuO₄, Jones reagent, Collins reagent and the like.

In an embodiment, oxidation in step (f) is performed in the presence of an organic solvent, e.g. DMSO or dichloromethane.

In an embodiment, oxidation in step (f) is performed by treatment with an oxidizing agent selected from IBX, DMP, PCC or PDC, and an organic solvent.

In an embodiment, oxidation in step (f) is carried out at a temperature between 0° C. and 80° C., such as between 10° C. and 70° C.

In an embodiment, halogenation in step (g) may be performed in the presence of a halide source and an acid.

Suitable halide sources include, among others, C₂, N-chlorosuccinimide, DCDMH, Br₂, N-bromosuccinimide, DBDMH, I₂, N-iodosuccinimide, DIDMH.

Suitable acids include, for example, HClO₄, MeSO₃H, p-ToISO₃H, CF₃SO₃H, PhCO₂H, CH₃CO₂H, PhSO₃H, HCl, HBr, HI, H₂SO₄, HNO₃, CF₃CO₂H, CCl₃CO₂H.

In an embodiment, halogenation in step (g) is performed in the presence of NBS or Br and an acid, such as HClO₄ or HBr.

In an embodiment, halogenation in step (g) is performed in the presence of an organic solvent, e.g. al alcohol, such as MeOH.

In an embodiment, oxidation in step (f) is carried out at a temperature between 0° C. and 80° C., such as between 10° C. and 50° C.

In an embodiment, reaction in step (h) may be performed in the presence of a base and a 5- to 10-membered heterocycle or a 5- to 10-membered heteroaryl, wherein the 5- to 10-membered heterocycle and the 5- to 10-membered heteroaryl are unsubstituted or substituted with C₁₋₆ alkyl, C₁₋₆ haloalkyl, halogen, —CN, NO₂, —N(Rₐ)(R_{b}), —ORr, —SR_{d}, —C(O)Rₑ, —C(O)OR_{f}, —C(O)N(R_{g})(Rₕ), —OC(O)Rᵢ; wherein Rₐ, R_{b}, R_{c}, R_{d}, Rₑ, R_{f}, R_{g}, Rₕ and Rᵢ are independently selected from hydrogen, C₁-C₆ alkyl and C₁-C₆ haloalkyl.

Suitable bases include inorganic and organic bases, such as an alkali metal carbonate or bicarbonate (e.g. Na₂CO₃, K₂CO₃, Cs₂CO₃, Li₂CO₃, NaHCO₃, KHCO₃, CsHCO₃, LiHCO₃), an alkali metal phosphate (e.g. Na₃PO₄, K₃PO₄, Na₂HPO₄, K₂HPO₄, NaH₂PO₄, KH₂PO₄), an alkali metal alkoxide (e.g. NaOMe, KOMe, NaOEt, KOEt, NaOtBu, KOtBu), an alkali metal hydroxide (e.g. NaOH, KOH, LiOH, CsOH), an aliphatic or aromatic amine (e.g. Me₂NH, Et₂NH, iPr₂NH, Bu₂NH, Me₃N, Et₃N, Bu₃N, iPr₂EtN, N-methylmorpholine, pyridine, DMAP, aniline, N,N-dimethylaniline). In an embodiment, the base is an inorganic base, such as an alkali metal carbonate, e.g. Na₂CO₃, K₂CO₃, Cs₂CO₃. In a particular embodiment, the base is K₂CO₃.

In an embodiment, the 5- to 10-membered heterocycle or 5- to 10-membered heteroaryl is a compound of formula

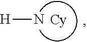

wherein Cy is selected from 5- to 10-membered heterocyclyl and 5- to 10-membered heteroaryl, wherein the 5- to 10-membered heterocyclyl and the 5- to 10-membered heteroaryl are unsubstituted or substituted with C₁₋₆ alkyl, C₁₋₆ haloalkyl, halogen, —CN, NO₂, —N(Rₐ)(R_{b}), —OR_{c}, —SR_{d}, —C(O)Rₑ, —C(O)OR_{f}, —C(O)N(R_{g})(Rₕ), —OC(O)Rᵢ; wherein Rₐ, R_{b}, R_{c}, R_{d}, Rₑ, R_{f}, R_{g}, Rₕ and Rᵢ are independently selected from hydrogen, C₁-C₆ alkyl and C₁-C₆ haloalkyl. In an embodiment, Rₐ, R_{b}, R_{c}, R_{d}, Rₑ, R_{f}, R_{g}, Rₕ and Rᵢ are independently selected from hydrogen, C₁-C₃ alkyl and C₁-C₃ haloalkyl.

According to an embodiment, the 5- to 10-membered heterocycle or 5- to 10-membered heteroaryl is selected from pyrrolidine, pyperidine, morpholine, piperazine, pyrrolepyrazole, imidazole, 1,2,3-triazole, 1,2,4-triazole, tetrazole, indole, isoindole, benzimidazole, indazole, benzotriazole, pyrazolo[3,4-b]pyridine, pyrazolo[3,4-c]pyridine, pyrazolo[4,3-b]pyridine, pyrazolo[4,3-c]pyridine, imidazo[4,5-b]pyridine, imidazo[4,5-c]pyridine, pyrazolo[3,4-d]pyrimidine, pyrazolo[4,3-d]pyrimidine, purine, pyrazolo[3,4-b]pyrazine, imidazo[4,5-b]pyrazine, benzotriazole, 1,2,3-triazolo[4,5-b]pyridine, 1,2,3-triazolo[4,5-c]pyridine, 1,2,3-triazolo[4,5-d]pyrimidine, 1,2,3-triazolo[4,5-b]pyrazine, wherein said compounds can be unsubstituted or substituted with C₁₋₆ alkyl, C₁₋₆ haloalkyl, halogen, —CN, NO₂, —N(Rₐ)(R_{b}), —OR_{c}, —SR_{d}, —C(O)Rₑ, —C(O)OR_{f}, —C(O)N(R_{g})(Rₕ), —OC(O)Rᵢ; wherein Rₐ, R_{b}, R_{c}, R_{d}, Rₑ, R_{f}, R_{g}, Rₕ and Rᵢ are independently selected from hydrogen, C₁-C₆ alkyl and C₁-C₆ haloalkyl.

In an embodiment, the substituent on the 5- to 10-membered heterocycle or 5- to 10-membered heteroaryl compound can be selected from C₁₋₃ alkyl, C₁₋₃ haloalkyl, halogen, —CN, NO₂, —N(Rₐ)(R_{b}), —OR_{c}, —SR_{d}, —C(O)Rₑ, —C(O)OR_{f}, —C(O)N(R_{g})(Rₕ), —OC(O)Rᵢ; wherein Rₐ, R_{b}, Rr, R_{d}, Rₑ, R_{f}, R_{g}, Rₕ and Rᵢ are independently selected from hydrogen, C₁-C₃ alkyl and C₁-C₃ haloalkyl. In a further embodiment, the substituent on the 5- to 10-membered heterocycle or 5- to 10-membered heteroaryl compound can be selected from Me, Et, CF₃, F, Cl, —CN, —NH₂, OMe, OEt, SMe, OCF₃, —COMe, —COOH, —COOMe, —CONH₂, —CONHMe, —CONMe₂.

In a particular embodiment, the 5- to 10-membered heterocycle or 5- to 10-membered heteroaryl compound is 4-cyanopyrazole, imidazole, 5-methyl-2H-tetrazole, 5-chloro-2H-benzotriazole or 1-piperazin-yl-ethanone. In an embodiment, it is 4-cyanopyrazole.

In an embodiment, reaction in step (h) is performed in the presence of an organic solvent, such as an aprotic organic solvent, e.g. DMSO, DMF, THF, CAN or acetone.

In an embodiment, reaction in step (h) is carried out at a temperature between 0° C. and 80° C., such as between 10° C. and 50° C.

In a particular embodiment, the invention is directed to a process for preparing zuranolone, or a salt or solvate thereof, which comprises (a) to (h) as disclosed herein wherein R¹ is H and R³ is 4-cyanopyrazole. In said embodiment, X and Y may be Br.

In an embodiment, the invention is directed to a process for preparing a compound of formula

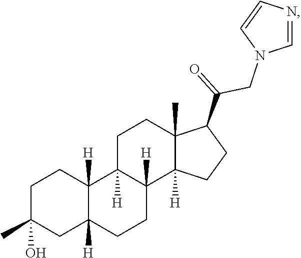

or a salt or solvate thereof, which comprises (a) to (h) as disclosed herein wherein R¹ is —H and R³ is imidazole. In said embodiment, X and Y may be Br.

In an embodiment, the invention is directed to a process for preparing a compound of formula

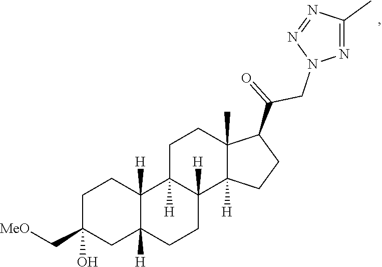

or a salt or solvate thereof, which comprises (a) to (h) as disclosed herein wherein R¹ is —OMe and R³ is 5-methyl-2H-tetrazole. In said embodiment, X and Y may be Br.

In an embodiment, the invention is directed to a process for preparing a compound of formula

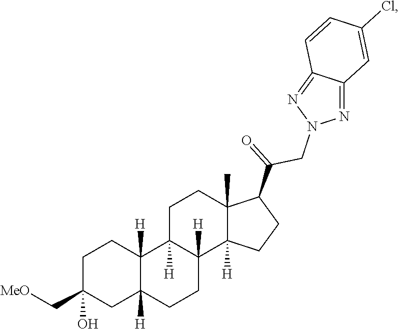

or a salt or solvate thereof, which comprises (a) to (h) as disclosed herein wherein R¹ is —OMe and R³ is 5-chloro-2H-benzotriazole. In said embodiment, X and Y may be Br.

In an embodiment, the invention is directed to a process for preparing a compound of formula

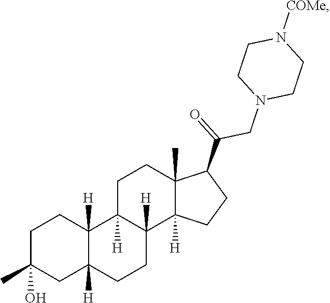

or a salt or solvate thereof, which comprises (a) to (h) as disclosed herein wherein R¹ is H and R³ is 1-piperazin-yl-ethanone. In said embodiment, X and Y may be Br.

Intermediate compounds Compounds of formula (I), and salts or solvates thereof, have been found by the inventors to be useful and versatile intermediates for the synthesis of active compounds, such as zuranolone, and intermediated thereof.

Therefore, in another aspect the invention is directed to a compound of formula (I) or a salt or solvate thereof

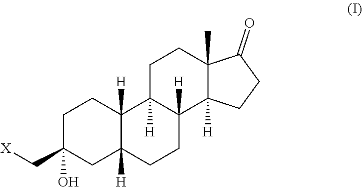

wherein X is selected from Cl, Br and I.

Compound of formula (III) is a useful intermediate in the preparation of compounds of formula (I) and, therefore, in the synthesis of zuranolone and structurally related compounds, such as those of formula (IX).

Therefore, in another aspect the invention is directed to a compound of formula (III), or a salt or solvate thereof

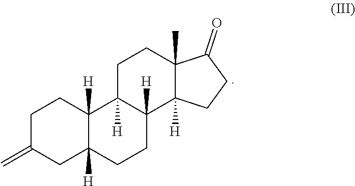

It should be understood that the scope of the present disclosure includes all the possible combinations of embodiments disclosed herein.

The following examples illustrate the invention, but are not intended to limit the scope of the invention.

### EXAMPLES

### Synthesis of Compound 2

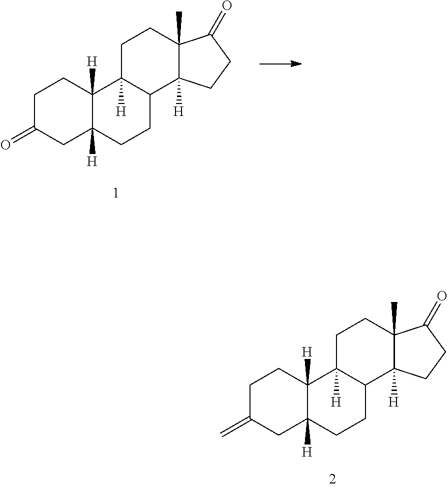

A suspension of MePPh₃Br (87.7 g, 24.05 mmol) and t-BuOK (27.0 g, 24.05 mmol) in THE (330 mL) was stirred for 1 hour at 20-25° C. under nitrogen atmosphere. The mixture was slowly added over a suspension 1 (60 g, 21.87 mmol) in THE (330 mL) at a temperature of 0-5° C. After consumption of starting material, water (600 mL) was added to the suspension, the solvent was removed under vacuum and the aqueous layer was extracted twice with DCM (300 mL). The replacement of DCM for heptane promotes phosphines precipitation. Compound 2 was isolated in MeOH/H₂O (white solid, 58.7 g, 98% yield). ¹H NMR (500 MHz, CDCl₃): 0.85 (s, 3H), 1.03-1.39 (m, 7H), 1.45-1.66 (m, 5H), 1.75-1.99 (m, 8H), 2.04 (dt, J=19.1, 8.6 Hz, 1H), 2.33 (t, J=13.3 Hz, 1H), 2.40 (ddd, J=19.1, 8.6, 0.82 Hz, 1H), 4.55 (s, 2H). ¹³C NMR (125 MHz, CDCl₃): 13.9, 21.8, 25.0, 25.2, 29.4, 29.5, 31.4, 31.8, 35.8, 36.0, 38.6, 38.8, 40.9, 41.4, 48.1, 50.7, 106.7, 150.2, 221.4.

### Synthesis of Compound 3

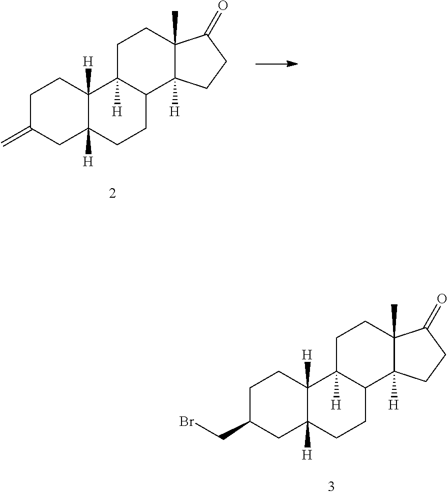

To a solution of 2 (38.1 g, 139.94 mmol) in acetone (495 mL), water (95 mL) was added and the reaction mixture was cooled down to −10/−15° C. Then, HClO₄ 70% (16.7 mL, 195.9 mmol) was added slowly without raising −10° C. Then, the temperature was adjusted at −10° C. and NBS (29.9 g, 167.92 mmol) was added in one portion. The reaction was stirred at that temperature until starting material consumption (92/8% isomer ratio, a/p). The reaction mixture was left to 10/15° C. Then, the reaction mixture was added to a solution of sodium metabisulfite (5% in H₂O, 580 mL). The obtained solid was filtered off and purified in a mixture isopropyl ether/heptane, obtaining 3 as a white solid (36.2 g, 70% yield, 97.5/2.5% isomer ratio α/β). ¹H NMR (500 MHz, CDCl₃): 0.89 (s, 3H), 1.06-1.41 (m, 7H), 1.48-1.54 (m, 2H), 1.54-1.59 (m, 4H), 1.67-1.85 (m, 5H), 1.87-1.98 (m, 3H), 2.07-2.14 (m, 2H), 2.45 (dd, J=19.3, 8.6 Hz, 1H), 3.70 (q, J=10.6 Hz, 2H). ¹³C NMR (125 MHz, CDCl₃): 13.9, 21.8, 24.9, 25.0, 25.1, 30.5, 31.1, 31.7, 34.4, 36.0, 37.0, 38.1, 40.2, 41.2, 44.1, 48.0, 50.7, 71.6, 221.4.

### Synthesis of Compound 4

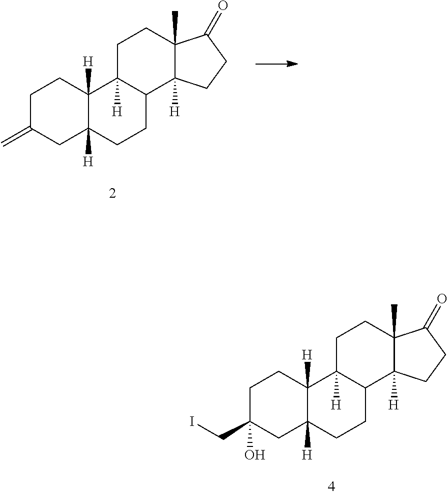

Over a solution of 2 (5 g, 18.2 mmol) in acetone (65 mL), water (12.5 mL) was added. The reaction mixture was cooled down to WOC and HClO₄ (2.65 mL, 31.0 mmol) was added without raising 5° C. Then, N-iodosuccinimide (6.17 g, 27.4 mmol) was added in one portion and the reaction was stirred at 0° C. for an hour. After consumption of the starting material, a solution of aqueous sodium metabisulfite was slowly added, and the reaction mixture was stirred for 10 minutes. Then, acetone was removed under vacuum and the aqueous phase was extracted with DCM. The aqueous phase was reextracted with another portion of DCM and the solvent removed under vacuum obtaining a yellow oil (79/21 α/β % isomer ratio). The compound was purified by column chromatography obtaining a white solid (4.9 g, 65% yield). ¹H NMR (500 MHz, CDCl₃): 0.83 (s, 3H), 1.02-1.35 (m, 7H), 1.41-1.58 (m, 6H), 1.61-1.76 (m, 4H), 1.79-1.96 (m, 4H), 1.99-2.08 (m, 2H), 2.41 (dd, J=19.2, 8.7 Hz, 1H), 3.54 (s, 2H). ¹³C NMR (125 MHz, CDCl₃): 13.9, 21.8, 22.3, 25.0, 25.1, 31.1, 31.4, 31.7, 34.4, 36.0, 37.9, 38.0, 40.2, 41.1, 48.0, 50.6, 70.9, 221.3.

### Synthesis of Compound 5

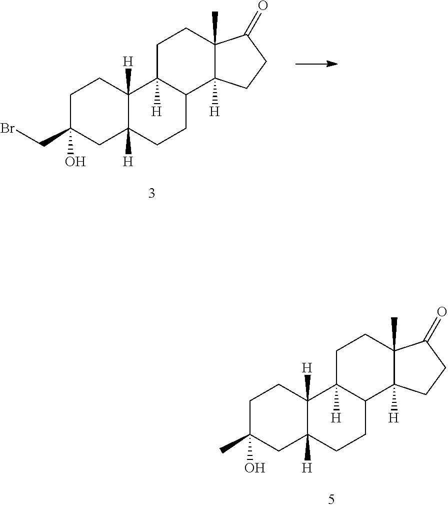

AIBN (1.38 g, 8.4 mmol) was added to a solution of 3 (34.5 g, 93.4 mmol) in THE (380 mL) under nitrogen atmosphere and then was heated at reflux. Then, Bu₃SnH (42.37 mL, 156 mmol) was added portionwise (4 portions). The reaction was stirred until starting material consumption (TLC: toluene/EtOAc, 4/1). Then, the reaction mixture was cooled at rt and the compound was isolated in heptane as a white solid (23.8 g, 69% yield, 99.3/0.7% isomer ratio).

¹H NMR (500 MHz, CDCl₃): 0.89 (s, 3H), 1.05-1.25 (m, 3H), 1.29 (s, 3H), 1.32-1.56 (m, 11H), 1.65-1.90 (m, 6H), 1.92-1.97 (m, 1H), 2.06-2.13 (m, 1H), 2.45 (dd, J=19.3, 8.6 Hz, 1H). ¹³C NMR (125 MHz, CDCl₃): 13.9, 21.8, 25.1, 25.3, 25.6, 26.6, 31.4, 31.8, 34.6, 34.9, 36.1, 38.2, 40.5, 41.2, 41.3, 48.1, 50.7, 72.1, 221.6.

### Synthesis of Compound 6

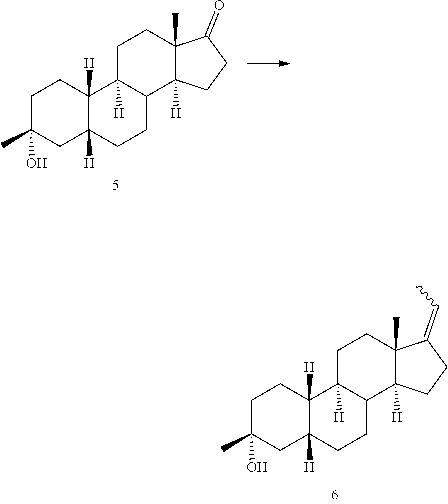

Over a suspension of ethyltriphenyl phosphonium bromide (165.4 g, 445 mmol) in THE (650 mL), potassium t-butoxide was added (50.1 g, 445 mmol). The mixture was heated at 60° C. and stirred for an hour. Then, a suspension of 5 (86.3 g, 297 mmol) in THE was added and the reaction mixture was stirred overnight at 60° C. After consumption of the starting material, the temperature was reduced until 20-25° C. and acetone (22 mL, 297 mmol) and water (860 mL) were added subsequently. Then, THE was evaporated under reduced pressure and DCM (860 mL) was added. The resulting aqueous layer was extracted with DCM (200 mL) and both organic layers were joined. The replacement of DCM for heptane promotes phosphines precipitation. 6 was not isolated and was used in solution in the next step.

### Synthesis of Compound 7

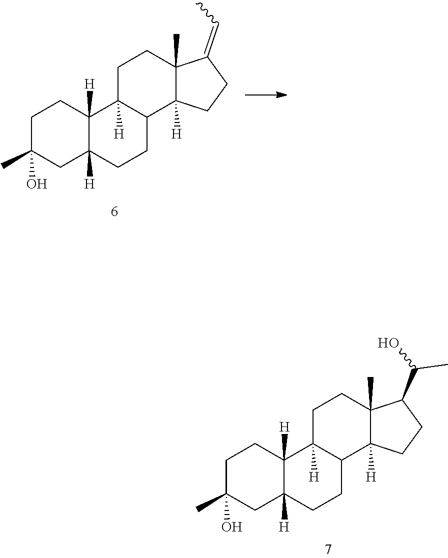

Over a solution of 6 (57.8 g, 191.1 mmol) in THE (752 mL), BH₃—SMe₂ (143.5 mL, 286.7 mmol) was charged through an addition funnel. The temperature was set at 20-25° C. and it was stirred for 3 h. When the boron intermediate was formed, water (289 mL) was charged slowly. Then, one part of sodium perborate (91.1, 573.3 mmol) was charged. The reaction was heated to 40° C. and it was stirred for 16 h. After positive control, the reaction was cooled to 20-25° C. and the salts were filtered. THE was eliminated and aqueous phase was extracted with methylene chloride (580 mL). The resulting aqueous phase was extracted with methylene chloride (290 mL). Organic phases were joined and DCM was changed for methanol. Water (175 mL) was charged. The reaction was cooled to 0/5° C. for 1 h and it was filtered obtaining a white solid (58.1 g, 94.9% yield).

### Synthesis of Compound 8

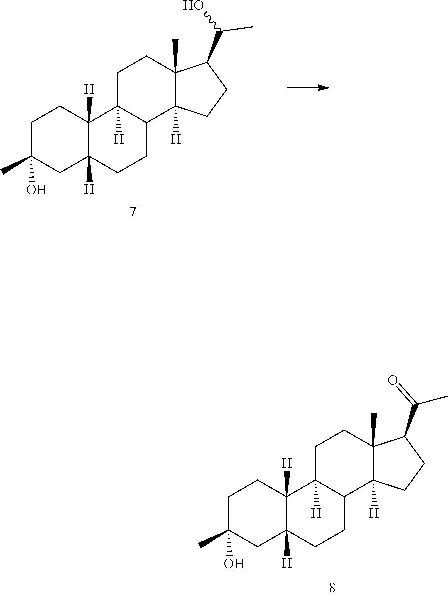

A suspension of IBX (7.54 g, 26.97 mmol) and 7 (7.86 g, 24.52 mmol) in DMSO (150 mL) was heated at 60° C. The reaction mixture was stirred at that temperature for an hour. After consumption of the starting material the mixture was cooled down to 20-25° C. Then, it was slowly added to a solution of sodium metabisulfite (7 g, 36.78 mmol) in water (160 mL) and stirred for an hour. The suspension was filtrated and washed with water. The solid was resuspended in water. The obtained solid was solved in DCM and was washed sequentially with a solution of NaHCO₃ and water. Then, the compound was precipitated in MeOH/water (5 g, 78.5% yield).

### Synthesis of Compound 9

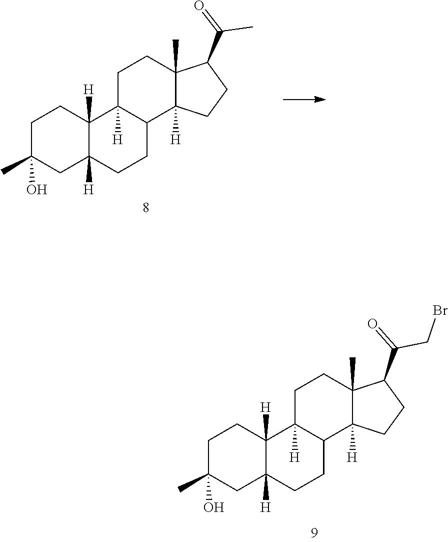

Over a suspension of 8 (5 g, 15.6 mmol) and NBS (3.35 g, 18.8 mmol) in MeOH (20 mL), HClO₄ (1.89 mL, 21.9 mmol) was added. The reaction mixture was stirred at 20-25° C. for 1 h 10 min. After consumption of starting material, MeOH (10 mL) and a solution of sodium metabisulfite (1.49 g, 7.84 mmol) in water (5 mL) were added, subsequently. Then, additional water was very slowly added, observing the precipitation of the product. The solid was filtrated and washed with water (5.79 g, 92.9% yield).

### Synthesis of Zuranolone

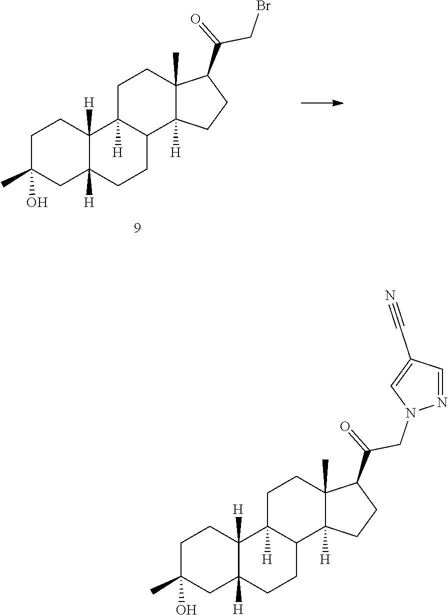

A suspension of 9 (16.5 g, 41.67 mmol), K₂CO₃ (7.14 g, 51.67 mmol) and 4-cyanopyrazole (4.62 g, 49.59 mmol) in DMSO (165 mL) was stirred for an hour at 20-25° C. After consumption of the starting material, the reaction mixture was slowly added to water (660 mL) and stirred for an hour. The suspension was filtrated and the solid washed with water (17 g, 99.8% yield). The compound was subsequently purified in MTBE and in EtOAc/heptane (13.7 g, 80.5% yield).

### Synthesis of Compound 10

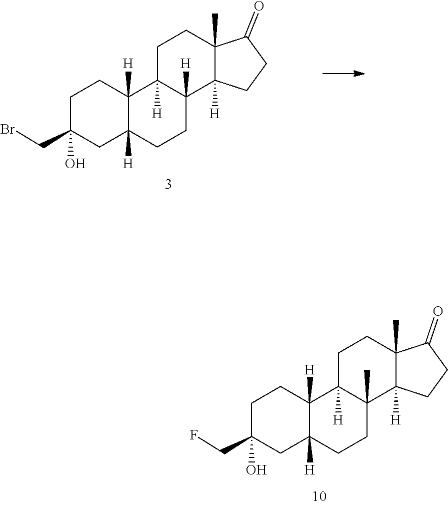

To a 250 mL flask with 3 (1.28 g, 3.4670 mmol), THF (50 mL) and K₂CO₃ (10 g) were added and the mixture heated to reflux for two days. It was cooled to rt, water was added, and it was extracted 3 times with ethyl acetate, concentrated in a flask of 100 mL, in which toluene (10 mL) and TBAF (50 mL solution 1 M in THF, 50 mmol) were added, it was concentrated at the rotary evaporator until a third of the volume, this operation was repeated twice, toluene (10 mL) and TBAF (40 mL 1 M solution in THF, 40 mmol) were added, concentrated at the rotary evaporator to one third of the volume, 20 mL of toluene were added and concentrated at the rotary evaporator to a final volume of 50 mL. It was heated to reflux (120° C.) for 72 hours, cooled to rt, water was added, extracted three times with ethyl acetate, dried with Na₂SO₄, filtered and concentrated. The obtained reaction crude (13.6 g), was purified by column chromatography, using heptane/ethyl acetate 3/1 as eluent, to provide 10 (660 mg, 62% yield).

¹H NMR (400 MHz, CDCl₃): 0.87 (s, 3H), 1.02-1.43 (m, 10H), 1.44-1.70 (m, 6H), 1.73-1.98 (m, 5H), 2.01-2.16 (m, 1H), 2.44 (dd, J=19.2, 8.6 Hz, 1H), 4.36 (q, J=9.4 Hz, 1H), 4.48 (q, J=9.4 Hz, 1H).

### Synthesis of Compound 11

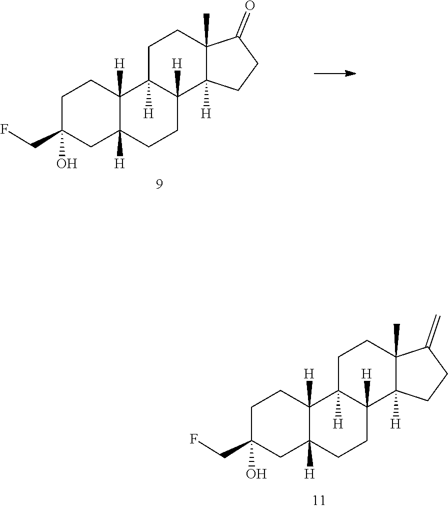

To a 50 mL flask, with MePPh₃Br (2.7 g, 7.6 mmol), three vacuum/Ar cycles were carried out, dry THE (20 mL) and t-BuOK (7.6 mmol, 7.6 mL, 1 M solution in THF) were added. The reaction turned yellow and was left under stirring for 1 hour at rt. 9 (586 mg, 1.9 mmol) dissolved in 5 mL of dry THE under Ar was added, and left under stirring for 15 hours at rt. Water was added, extracted with ethyl acetate three times, dried with Na₂SO₄, filtered and concentrated. The obtained reaction crude (2.1 g) was purified by column chromatography, using as eluent heptane/ethyl acetate 8/1, resulting in compound 11 (55 mg, 9% yield). ¹H NMR (400 MHz, CDCl₃): 0.79 (s, 3H), 1.08-1.16 (m, 4H), 1.18-1.35 (10H), 1.57-1.85 (9H), 2.18-2.30 (m, 2H), 2.47 (m, 1H), 4.48 (dq, 2H), 4.62 (m, 2H).

### Synthesis of Compound 12

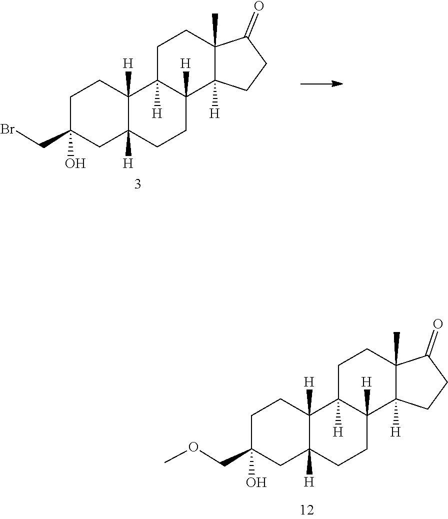

To a 100 mL flask with 3 (1 g, 2.70 mmol), was added methanol (50 mL), 25% MeONa (2.5 mL, 10.83 mmol) and heated at reflux for 29 hours. The mixture was allowed to cool to rt, water was added and extracted twice with DCM, dried with Na₂SO₄, filtered and concentrated. The yellow oil obtained does not need purification, leading directly to product 12 (782 mg, 90% yield). ¹H NMR (400 MHz, CDCl₃): 0.85 (s, 3H), 1.01-1.15 (m, 2H), 1.18-1.57 (m, 11H), 1.59-1.67 (m, 2H), 1.73-1.77 (m, 3H), 1.79-1.85 (m, 2H), 1.90-1.95 (m, 1H), 2.07 (dt, J=19.2, 8.9 Hz, 1H), 2.43 (dd, J=19.2, 8.6 Hz, 1H) 3.35-3.45 (m, 5H).

### Synthesis of Compound 13

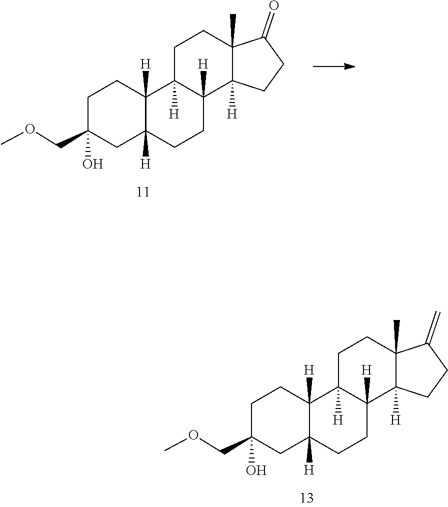

In a 25 mL flask, containing MePPh₃Br (812 mg, 2.27 mmol) and t-BuOK (255 mg, 2.27 mmol), three vacuum/Ar cycles were made, dry THE (7 mL) was added and left under stirring for 1 hour at rt. Compound 11 (607 mg, 1.89 mmol) placed under inert Ar atmosphere in 3 mL of dry THE was added over the previously prepared ylide. It was left under stirring at rt for 22 hours and a saturated aqueous solution of NH₄Cl was added, extracted with ethyl acetate three times, dried with Na₂SO₄, filtered and concentrated. The obtained reaction crude (1.1 g), was purified by column chromatography, using as eluent heptane/ethyl acetate 4/1, leading to compound 13 (270 mg, 45% yield). ¹H NMR (400 MHz, CDCl₃): 0.77 (s, 3H), 1.06-1.88 (m, 21H), 2.23 (m, 1H), 2.46-2.51 (m, 1H), 3.37-3.43 (m, 5H), 4.62 (m, 2H).

### Synthesis of Compound 14

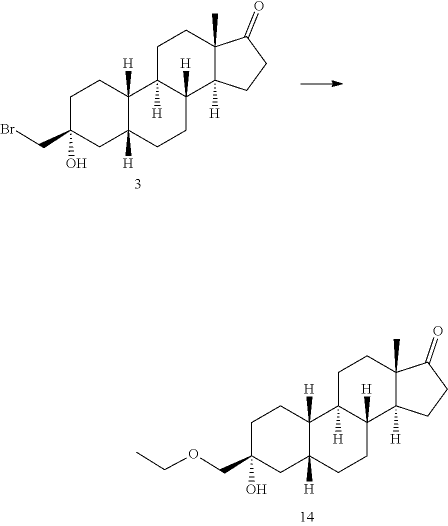

To a 100 mL flask, with 3 (1 g, 2.70), was added absolute EtOH (50 mL) and 20% EtONa (13.5 mmol, 4.9 mL) and heated to reflux for 5 hours. The mixture was cooled to rt and a saturated aqueous solution of NH₄Cl was added, extracted with ethyl acetate twice, dried with Na₂SO₄, filtered and concentrated. The obtained reaction crude (1.03 g), was purified by column chromatography, using as eluent heptane/ethyl acetate 1.5/1, leading to product 14 (561 mg, 62% yield). ¹H NMR (400 MHz, CDCl₃): 0.82 (s, 3H), 1.15-1.92 (m, 24H), 2.04 (dt, J=19.2, 8.9 Hz 1H), 2.38 (dd, J=19.3, 8.4 Hz, 1H), 3.38 (AB system, J=9.2 Hz, 2H), 3.49 (q, J=9.2 Hz, 2H).

### Synthesis of Compound 15

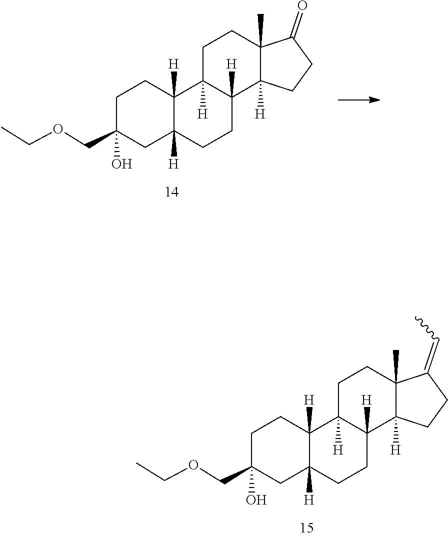

To a flask, with EtPPh₃Br (444 mg, 1.2 mmol), three vacuum/Ar cycles were made, added dry THE (2 mL) and t-BuOK (1.2 mmol, 1.2 mL, in 1 M THF) and leave to stir 1 hour at rt. A solution of 14 (200 mg, 0.5979 mmol) in THE under inert Ar atmosphere was added, and left to stir at rt for 3 days. Water was added to the reaction, and extracted with ethyl acetate twice, dried with Na₂SO₄, filtered and concentrated. The reaction crude (533 mg) was purified by column chromatography, using as eluent heptane/ethyl acetate 6/1, leading to compound 15 (153 mg, 73% yield). ¹H NMR (400 MHz, CDCl₃): 0.86 (s, 3H), 1.04-1.84 (m, 26H), 2.13-2.25 (m, 2H), 2.32-2.37 (m, 1H), 3.41 (AB system, J=9.2 Hz, 2H), 3.52 (q, J=7.0 Hz, 2H), 5.10 (m, 1H).

### Synthesis of Compound 16

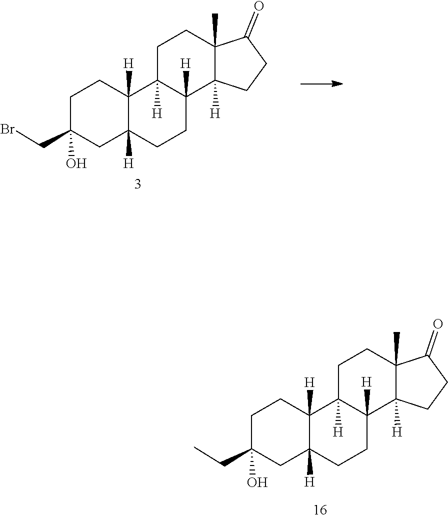

To a flask with CuI (2.1 g, 10.8 mmol) and large stirring core, three vacuum/Ar cycles were made and dry ether (70 mL) was added. The mixture was cooled to −30° C. and MeLi (21.7 mmol, 13.5 mL in 1.6 M ether solution) was added dropwise. It was stirred for 30 min at −30° C., lowered to −78° C., and 3 (1.0 g, 2.7 mmol in 10 mL solution of dry THF, under Ar) was added dropwise. It was allowed to stir for 2 h at −78° C., the flask was opened and 28% NH₃ in water was added, allowed to rise in temperature and saturated aqueous solution of NH₄Cl was added, extracted twice with DCM, cleaned the organic phases with NH₄Cl (aq), dried with Na₂SO₄, filtered and concentrated. The obtained reaction crude (760 mg), was purified by column chromatography, using as eluent heptane/methyl tertiary-butyl ether 1/1, leading to 16 (350 mg, 42% yield). ¹H NMR (400 MHz, CDCl₃): 0.85-0.88 (m, 6H), 1.03-1.81 (m, 22H), 1.88-1.95 (m, 1H), 2.06 (dt, J=19.2, 8.9 Hz, 1H), 2.41 (dd, J=11.2, 8.0 Hz, 1H).

### Synthesis of Compound 17

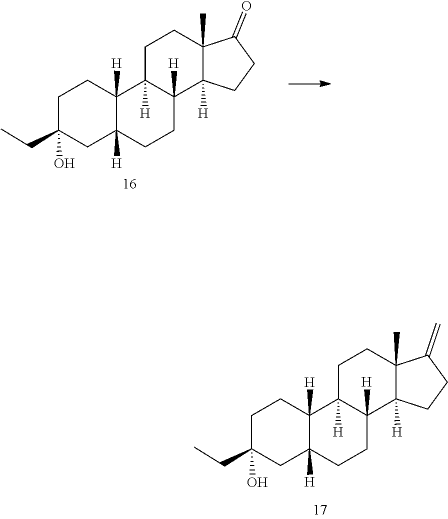

To a 25 mL flask with MePPh₃Br (714 mg, 2.0 mmol), three vacuum/Ar cycles were made, dry THF (2 mL) and t-BuOK (2.0 mmol, 2 mL, 1 M solution in THF) were added. The reaction turned yellow and it was left under stirring for 1 h at rt. 16 (160 mg, 0.55 mmol) dissolved in 2 mL of dry THF under Ar was added, and left under stirring for 15 h at rt. Water was added, extracted with ethyl acetate three times, dried with Na₂SO₄, filtered and concentrated. The obtained reaction crude (527 mg), was purified by column chromatography, using as eluent heptane/ethyl acetate 6/1, leading to compound 17 (102 mg, 64% yield). ¹H NMR (400 MHz, CDCl₃): 0.77 (s, 3H), 0.87 (t, J=7.6 Hz, 3H), 1.04-1.16 (m, 4H), 1.20-1.48 (12H), 1.57-1.85 (7H), 2.18-2.66 (m, 1H), 2.47 (m, 1H), 4.60-4.62 (m, 2H).

### Synthesis of Compound 18

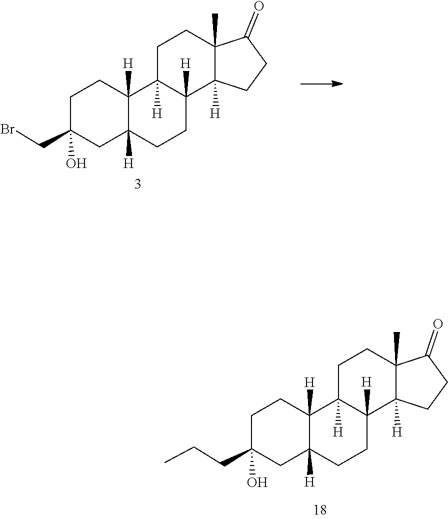

To a flask with CuI (2.1 g, 10.8 mmol) and large stirring core, three vacuum/Ar cycles were made and dry ether (70 mL) was added. The mixture was cooled to −30° C., and EtLi (21.7 mmol, 43.3 mL in 0.5 M benzene solution) was added dropwise. It was stirred for 30 min at −30° C., lowered to −78° C., and 3 (1.0 g, 2.7 mmol in 10 mL solution of dry THF, under Ar) was added dropwise. It was left to stir for 3.5 h at −78° C., the flask was opened and NH₃ 28% in water was added, the temperature was allowed to rise and a saturated aqueous solution of NH₄Cl was added, extracted twice with AcOEt, the organic phases were cleaned with NH₄Cl (ac), dried with Na₂SO₄, filtered and concentrated. The obtained reaction crude (1.04 g) was purified by column chromatography, using as eluent heptane/methyl tertiary butyl ether 1/1, leading to 18 (428 mg, 50% yield). ¹H NMR (400 MHz, CDCl₃): 0.86 (s, 3H), 0.92 (t, J=7.2 Hz, 3H), 1.04-1.84 (m, 24H), 1.89-1.95 (m, 1H), 2.07 (dt, J=19.3, 8.7 Hz, 1H), 2.42 (dd, J=19.3, 8.7 Hz, 1H).

### Synthesis of Compound 19

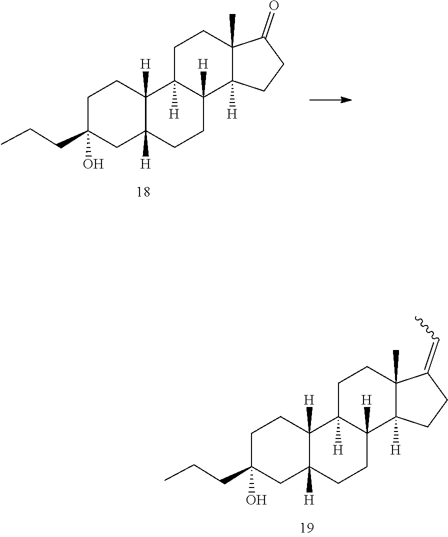

To a flask with EtPPh₃Br (280 mg, 0.7535 mmol), three vacuum/Ar cycles were made, was added dry THF (2 mL) and t-BuOK (0.7535 mmol, 0.75 mL, in 1 M THF) and left to stir for 1 h at rt. A solution of 18 (120 mg, 0.3768 mmol) in THF under inert Ar atmosphere was added, and left to stir at rt for 3 days. Water was added to the reaction, and extracted with ethyl acetate twice, dried with Na₂SO₄, filtered and concentrated. The reaction crude (256 mg), was purified by column chromatography, using as eluent heptane/ethyl acetate 6/1, leading to compound 19 (46 mg, 37% yield). ¹H NMR (500 MHz, CDCl₃): 0.87 (s, 3H), 0.93 (t, J=7.3 Hz, 3H), 1.02-1.39 (m, 16H), 1.41-1.50 (m, 4H), 1.51-1.56 (m, 2H), 1.65-1.86 (m, 6H), 2.12-2.27 (m, 2H), 2.31-2.40 (m, 1H), 5.11 (dt, J=4.2, 2.5 Hz, 1H).

### Synthesis of Compound 20

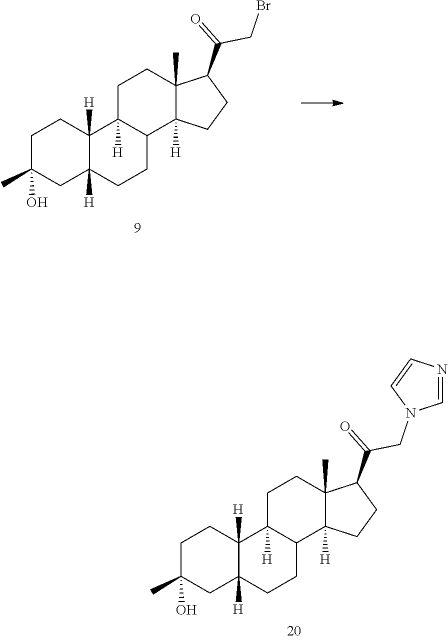

A suspension of 9 (1 g, 2.51 mmol), K₂CO₃ (0.174, 1.25 mmol) and imidazole (0.174 g, 2.56 mmol) in THF (10 mL) was stirred for six hours at reflux. After consumption of the starting material, the reaction mixture was cooled down to 20-25° C. Then, water was slowly added and the mixture stirred for 10 minutes. Tetrahydrofuran was removed under vacuum and the product was extracted with DCM (15 mL). The solvent was removed under vacuum (0.95 g, 99% yield). ¹H (500 MHz, CDCl₃): 0.85 (s, 3H), 1.03-1.39 (m, 10H), 1.45-1.66 (m, 6H), 1.75-1.99 (m, 8H), 2.04 (m, 1H), 2.10 (m, 1H), 2.59 (t, J=10.0 Hz, 1H), 4.76 (d, J=5.0 Hz, 2H), 6.88 (s, 1H), 7.08 (s, 1H), 7.57 (s, 1H). ¹³C (125 MHz, CDCl₃): 14.0, 23.3, 24.3, 24.4, 25.7, 26.1, 31.4, 31.5, 34.7, 38.9, 39.3, 40.3, 41.2, 41.7, 45.4, 55.8, 55.9, 58.8, 61.1, 72.0, 123.1, 128.6, 138.5, 203.3.

### Comparative Example 1

Addition of MeMgCl to compound 1 following the process disclosed for example in WO2020/118060 (synthesis of A25 in said document) was performed. However, this strategy led to formation of the β isomer (not desired) instead of the α-isomer. When a lower amount of MeMgCl was used (6 equivalent) the reaction was not completed. For this reason, 10 equivalents of MeMgCl were used in the example shown below.

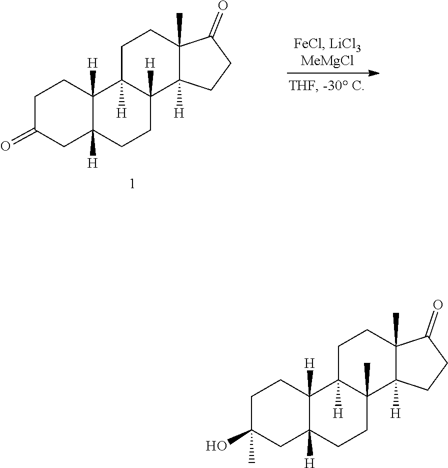

Over a solution of LiCl (0.32 g, 7.65 mmol) in THF (15 mL), FeCl₃ (0.48 g, 3.1 mmol) was added. The reaction mixture was cooled down to −30° C. and MeMgCl (3M, 6.1 mL) was slowly added without raising −20° C. After stirring the mixture for 30 min, 1 (0.5 g, 1.82 mmol) was added in one portion. After consumption of the starting material, the reaction mixture was heated until 10° C. and 10% citric acid (1 mL) was slowly added. EtOAc (10 mL) was added and the mixture was stirred for 15 min. Then, another portion of 10% citric acid (11 ml) was added and the mixture stirred until complete dissolution. After decantation, the organic phase was washed with brine (10 mL), and the solvent removed under reduced pressure, obtaining a white solid (0.5 g, β isomer).

### Comparative Example 2

Addition of MeMgCl to compound 1 following the process similar to WO2014/169832 (synthesis of SA-C in said document) was performed. However, this strategy led to a mixture of four compounds. When the reaction was performed in toluene at −78° C., as in the prior art, the reaction mixture frozen. The same happened at −10° C. For this reason, a mixture of toluene ant THF at −10° C. was used in the example shown below.

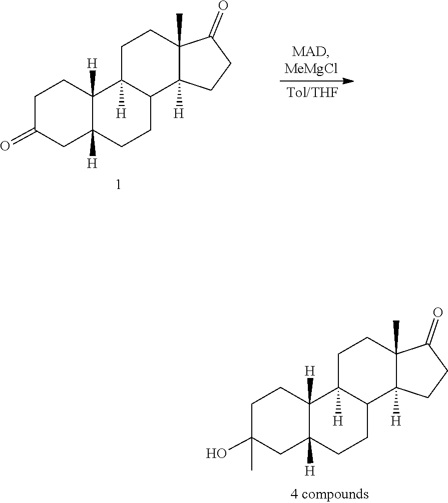

Preparation of MAD: Over a solution of 2,6-ditertbutyl-4-methyl phenol (2.04 g, 10.94 mmol) in toluene (3 mL) at 0/5° C., AlMeₛ(2.8 mL, 5.46 mmol) was added dropwise. The reaction mixture was heated at 20-25° C. for an hour.

Then, a solution of 1 (0.5 g, 1.82 mmol) in toluene/THF (3 mL/0.5 mL) was added and the reaction mixture was cooled down to −10° C. and stirred for 15 minutes. MeMgCl (1.9 mL, 5.46 mmol) was added observing a thick slurry (the temperature was readjusted to −5° C. observing a better stirring). Different reaction controls were taken, observing the formation of four compounds.

Similar results were obtained when using MeMgCl in the absence of MAD.

1. A process for preparing a compound of formula (I) or a salt or solvate thereof 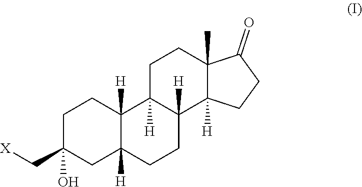
wherein
X is selected from Cl, Br and I;
which comprises
(a) olefination of a compound of formula (II) or a salt or solvate thereof 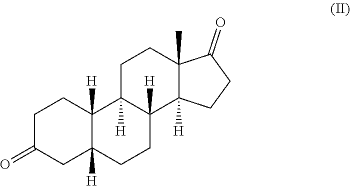
to provide a compound of formula (III) or a salt or solvate thereof 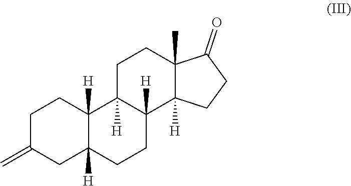
and
(b) halohydrin formation reaction of the compound of formula (III), or a salt or solvate thereof, to provide the compound of formula (I), or a salt or solvate thereof.

2. Process according to claim 1, wherein step (a) comprises reaction of the compound of formula (II), or a salt or solvate thereof, with a compound of formula (X) 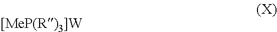
wherein
W is halogen; and
each R″ is independently selected from C₆-C₁₀ aryl;
in the presence of a base.

3. Process according to claim 1, wherein step (b) comprises reaction of a compound of formula (III), or a salt or solvate thereof, with a halohydrin forming reagent selected from HOCl, HOBr, HOI, N-chlorosuccinimide in the presence of water, N-bromosuccinimide in the presence of water, N-iodosuccinimide in the presence of water, Cl₂ in the presence of water, Br₂ in the presence of water, I₂ in the presence of water, 1,3-dichloro-5,5-dimethylhydatoin (DCDMHj in the presence of water, 1,3-dibromo-5,5-dimethylhydatoin (DBDMH) in the presence of water, and 1,3-diiodo-5,5-dimethylhydatoin (DIDMH) in the presence of water.

4. Process according to claim 1, wherein X is selected from Br and I.

5. Process according to claim 1, which further comprises
(c) conversion of the compound of formula (I), or a salt or solvate thereof, into a compound of formula (IV), or a salt or solvate thereof 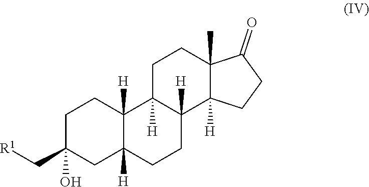
wherein
R¹ is selected from H, F, C₁₋₆ alkyl, C₁₋₆ alkoxyl, C₁₋₆ haloalkyl, and N(R′)₂, wherein each R′ is independently selected from H and C₁₋₆ alkyl.

6. Process according to claim 5, wherein step (c) comprises dehalogenation of the compound of formula (I), or a salt or solvate thereof, to provide a compound of formula (IV) wherein R¹ is H, or a salt or solvate thereof.

7. Process according to claim 5, wherein step (c) comprises reaction of the compound of formula (I), or a salt or solvate thereof, with a base and then with a fluoride source to provide a compound of formula (IV) wherein R¹ is F, or a salt or solvate thereof.

8. Process according to claim 5, wherein step (c) comprises reaction of the compound of formula (I), or a salt or solvate thereof, with a compound of formula (C₁₋₆ alkyl)MgZ, wherein Z is selected from Cl, Br, I, or with a compound of formula (C₁₋₆ alkyl)₂CuLi, to provide a compound of formula (IV) wherein R¹ is C₁₋₆ alkyl, or a salt or solvate thereof.

9. Process according to claim 5, wherein step (c) comprises reaction of the compound of formula (I), or a salt or solvate thereof, with a compound of formula (C₁₋₆ alkoxyl)M, wherein M is selected from Na and K, to provide a compound of formula (IV) wherein R¹ is C₁₋₆ alkoxyl, or a salt or solvate thereof.

10. Process according to claim 5, wherein step (c) comprises reaction of the compound of formula (I), or a salt or solvate thereof, with a compound of formula (C₁₋₆ haloalkyl)MgZ, wherein Z is selected from Cl, Br, I, or with a compound of formula (C₁₋₆ haloalkyl)₂CuLi, to provide a compound of formula (IV) wherein R¹ is C₁₋₆ haloalkyl, or a salt or solvate thereof.

11. Process according to claim 5, wherein step (c) comprises reaction of the compound of formula (I), or a salt or solvate thereof, with a compound of formula HN(R′)₂, wherein each R′ is independently selected from H and C₁₋₆ alkyl, to provide a compound of formula (IV) wherein R¹ is N(R′)₂, wherein each R′ is independently selected from H and C₁₋₆ alkyl, or a salt or solvate thereof.

12. Process according to claim 5, which further comprises
(d) olefination of the compound of formula (IV), or a salt or solvate thereof, to provide a compound of formula (V), or a salt or solvate thereof 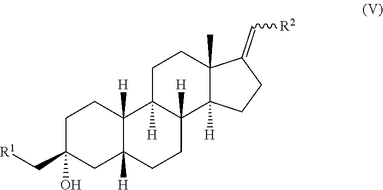
wherein
R¹ is selected from H, F, C₁₋₆ alkyl, C₁₋₆ alkoxyl, C₁₋₆ haloalkyl, and N(R′)₂, wherein each R′ is independently selected from H and C₁₋₆ alkyl; and
R² is selected from H and C₁₋₆ alkyl.

13. Process for preparing a compound of formula (IX) or a salt or solvate thereof 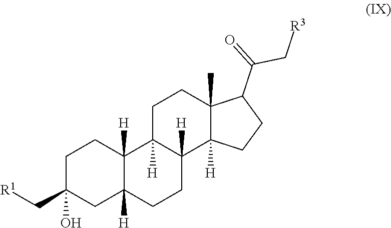
wherein
R¹ is selected from H, F, C₁₋₆ alkyl, C₁₋₆ alkoxyl, C₁₋₆ haloalkyl, and N(R′)₂, wherein each R′ is independently selected from H and C₁₋₆ alkyl; and
R³ is selected from 5- to 10-membered heterocyclyl and 5- to 10-membered heteroaryl, wherein the 5- to 10-membered heterocyclyl and the 5- to 10-membered heteroaryl are unsubstituted or substituted with a substituent selected from the group consisting of C₁₋₆ alkyl, C₁₋₆ haloalkyl, halogen, —CN, NO₂, —N(Rₐ)(Rₕ), —OR_{c}, —SR_{d}, —C(O)Rₑ, —C(O)OR_{f}, —C(O)N(R_{g})(Rₕ) and —OC(O)Rᵢ; wherein Rₐ, R_{b}, R_{c}, R_{d}, Rₑ, R_{f}, R_{g}, Rₕ and Rᵢ are independently selected from hydrogen, C₁-C₆ alkyl and C₁-C₆ haloalkyl;
which comprises
(a) olefination of a compound of formula (II) or a salt or solvate thereof 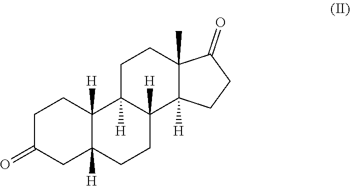
to provide a compound of formula (III) or a salt or solvate thereof 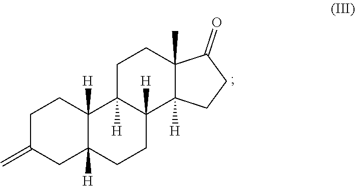
(b) halohydrin formation reaction of the compound of formula (III), or a salt or solvate thereof, to provide the compound of formula (I), or a salt or solvate thereof, 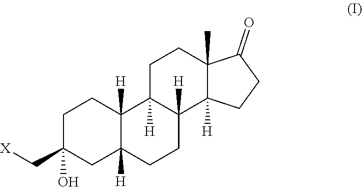
wherein X is selected from Cl, Br and I;
(c) conversion of the compound of formula (I), or a salt or solvate thereof, into a compound of formula (IV), or a salt or solvate thereof 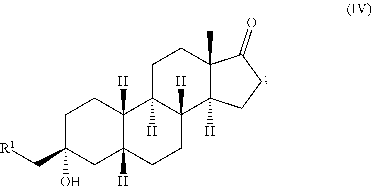
(d) olefination of the compound of formula (IV), or a salt or solvate thereof, to provide a compound of formula (Va), or a salt or solvate thereof 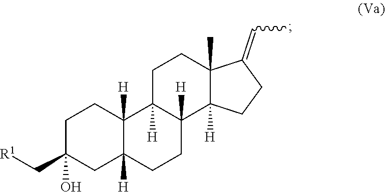
(e) hydroxylation of the compound of formula (Va), or a salt or solvate thereof, to provide a compound of formula (VI), or a salt or solvate thereof 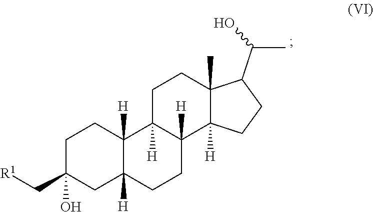
(f) oxidation of the compound of formula (VI), or a salt or solvate thereof, to provide a compound of formula (VII), or a salt or solvate thereof 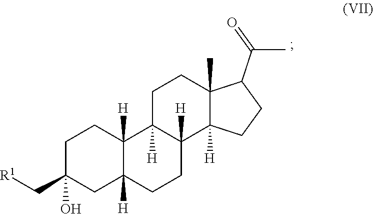
(g) halogenation of the compound of formula (VII), or a salt or solvate thereof, to provide a compound of formula (VIII), or a salt or solvate thereof 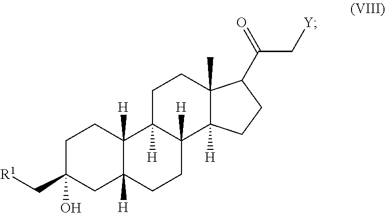
wherein Y is halogen; and
(h) reaction of the compound of formula (VIII), or a salt or solvate thereof, with a 5- to 10-membered heterocycle or a 5- to 10-membered heteroaryl, wherein the 5- to 10-membered heterocycle and the 5- to 10-membered heteroaryl are unsubstituted or substituted with a substituent selected from the group consisting of C₁₋₆ alkyl, C₁₋₆ haloalkyl, halogen, —CN, NO₂, —N(Rₐ)(R_{b}), —OR_{c}, —SR_{d}, —C(O)Rₑ, —C(O)OR_{f}, —C(O)N(R_{g})(Rₕ) and —OC(O)R; wherein Rₐ, R_{b}, R_{c}, R_{d}, Rₑ, R_{f}, R_{g}, Rₕ and Rᵢ are independently selected from hydrogen, C₁-C₆ alkyl and C₁-C₆ haloalkyl;
to provide a compound of formula (IX) or a salt or solvate thereof.

14. Process according to claim 13, wherein the compound of formula (IX) is zuranolone, or a salt or solvate thereof.

15. A compound selected from 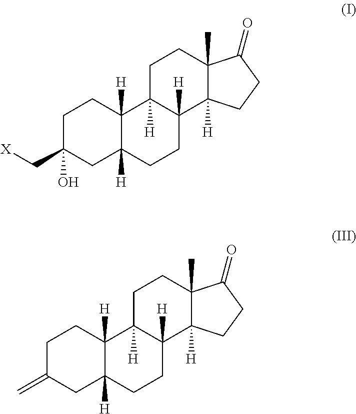
or a salt or solvate thereof, wherein X is selected from Cl, Br and I.

16. Process according to claim 2, wherein W is Br.

17. Process according to claim 2, wherein each R″ is phenyl.

18. Process according to claim 1, wherein X is Br.
